SEQUENCE LISTING (25 sequences)

SEQ ID NO: 1 - protein, 754 aa, Homo sapiens
MSPEPVPPPPPPQCPGCDRAEPIAQRLEEGDEAFRAGDYEMAAELFRSMLAGLAQPDRGLCLRLGDALARAGRLPEALGAFRGAARLGALRPEELEELAGGLVRAVGLRDRPLSAENPGGEPEAPGEGGPAPEPRAPRDLLGCPRCRRLLHKPVTLPCGLTVCKRCVEPGPARPQVRRVNVVLSGLLEKCFPAECRLRRLAGQARSLQRQQQPEAALLRCDQALELAPDDNSLLLLRAELYLTMKNYEQALQDASAACQNEPLLIKGHQVKAQALSGLGRSKEVLKEFLYCLALNPECNSVKKEAQKVMCEVLFSATANVHENLTSSIQSRLKAQGHSHMNAQALLEEGDAGSSENSSEKSDMLGNTNSSVLYFILGLHFEEDKKALESILPTAPSAGLKRQFPDDVEDAPDLNAPGKIPKKDLSLQRSPNSETEESQGLSLDVTDFECALCMRLLFEPVTTPCGHTFCLKCLERCLDHAPHCPLCKDKLSELLASRNFNITVLAEELIFRYLPDELSDRKRIYDEEMSELSNLTRDVPIFVCAMAFPTVPCPLHVFEPRYRLMIRRCMETGTKRFGMCLSAEHAGLSEYGCMLEIKDVRTFPDGSSVVDAIGISRFRVLSHRHRDGYNTADIEYLEDEKVEGPEYEELAALHDSVHQQSVSWFASLQDRMKEQILSHFGVMPDREPEPQSNPSGPAWSWWILAVLPLERKAQLAILGMTSLKERLLAIRRILVIITRKMNSRQELANARERNN

SEQ ID NO: 2 - protein, 201 aa, Homo sapiens
VPIFVCAMAFPTVPCPLHVFEPRYRLMIRRCMETGTKRFGMCLSAEHAGLSEYGCMLEIKDVRTFPDGSSVVDAIGISRFRVLSHRHRDGYNTADIEYLEDEKVEGPEYEELAALHDSVHQQSVSWFASLQDRMKEQILSHFGVMPDREPEPQSNPSGPAWSWWILAVLPLERKAQLAILGMTSLKERLLAIRRILVIITR

SEQ ID NO: 3 - protein, 550 aa, Photinus pyralis
MEDAKNIKKGPAPFYPLEDGTAGEQLHKAMKRYALVPGTIAFTDAHIEVNITYAEYFEMSVRLAEAMKRYGLNTNHRIVVCSENSLQFFMPVLGALFIGVAVAPANDIYNERELLNSMNISQPTVVFVSKKGLQKILNVQKKLPIIQKIIIMDSKTDYQGFQSMYTFVTSHLPPGFNEYDFVPESFDRDKTIALIMNSSGSTGLPKGVALPHRTACVRFSHARDPIFGNQIIPDTAILSVVPFHHGFGMFTTLGYLICGFRVVLMYRFEEELFLRSLQDYKIQSALLVPTLFSFFAKSTLIDKYDLSNLHEIASGGAPLSKEVGEAVAKRFHLPGIRQGYGLTETTSAILITPEGDDKPGAVGKVVPFFEAKVVDLDTGKTLGVNQRGELCVRGPMIMSGYVNNPEATNALIDKDGWLHSGDIAYWDEDEHFFIVDRLKSLIKYKGYQVAPAELESILLQHPNIFDAGVAGLPDDDAGELPAAVVVLEHGKTMTEKEIVDYVASQVTTAKKLRGGVVFVDEVPKGLTGKLDARKIREILIKAKKGGKSKL

SEQ ID NO: 4 - protein, 550 aa, Photinus pyralis
MEDAKNIKKGPAPFYPLEDGTAGEQLHKAMKRYALVPGTIAFTDAHIEVNITYAEYFEMSVRLAEAMKRYGLNTNHRIVVCSENSLQFFMPVLGALFIGVAVAPANDIYNERELLNSMNISQPTVVFVSKKGLQKILNVQKKLPIIQKIIIMDSKTDYQGFQSMYTFVTSHLPPGFNEYDFVPESFDQDKTIALIMNSSGSTGLPKGVALPHRTACVRFSHARDPIFGNQIIPDTAILSVVPFHHGFGMFTTLGYLICGFQVVLMYRFEEELFLRSLQDYKIQSALLVPTLFSFFAKSTLIDKYDLSNLHEIASGGAPLSKEVGEAVAKRFHLPGIRQGYGLTETTSAILITPEGDDKPGAVGKVVPFFEAKVVDLDTGKTLGVNQRGELCVRGPMIMSGYVNNPEATNALIDKDGWLHSGDIAYWDEDEHFFIVDRLKSLIKYKGYQVAPAELESILLQHPNIFDAGVAGLPDDDAGELPAAVVVLEHGKTMTEKEIVDYVASQVTTAKKLRGGVVFVDEVPKGLTGKLDARKIREILIKAKKGGKSKL

SEQ ID NO: 5 - protein, 754 aa, Mus musculus
MSPEPAPPEPQGVRRSLEEPPEEPQGQGSAAFLAGDYEVAAELFRSLLAGLAQPERGLCLQLGEALARAGRLPEAIGAFRGAALLGPLQPGELSELASGLARALGLREKRPPVAARPSGCASGAPGPVAPRDLLGCPRCGRLLYKPVTLSSGLTVCKRCVEQVPVRAQARRVNVMLSGLLERCFPAECRLRRLACQARGLHRQQQPEAALLRCQQALDMAPDDNSLLLLRAELYLTMKNYDQALQDAEAVCQREPLLTKGHHIKAQILSGLGRHREVLKEFIYCLALNPECNSAKKETQKVICEVFFSTSESEHQTSTSSTETGPEALCEGQTNPQYPLEEAGGHANADNPKTPSEKSDAPADINSSVLYFILGLHCEEDKKALEGIVPAAPSSTLKRQLPSDAQDDEELKANTPEKIPKKDADSPPQRNASSLEEEPEFTIDATDFECALCMRLLFEPVTTPCGHTFCLKCLERCLDHAPHCPLCKDKLSELLATRNFNVTVLTEELIFRYLPDELSDRKRVYDEEMSELSNLTRDVPIFVCAMAFPTVPCPLHVFEPRYRLMIRRCMETGTKRFGMCLSAENAGISEYGCMLEIKDVRTFPDGSSVVDAIGISRFRVLSHRHRDGYNTADIEYLEDEKVEGPEFEELTALHESVYQQSVSWFASLQDHMKKQILSHFGSMPDREPEPQSNSSGPAWSWWILAVLPLERKAQLAILGMASLKERLLAIRRILVIITRKLNSRQEMANNTQRDN

SEQ ID NO: 6 - protein, 201 aa, Mus musculus
VPIFVCAMAFPTVPCPLHVFEPRYRLMIRRCMETGTKRFGMCLSAENAGISEYGCMLEIKDVRTFPDGSSVVDAIGISRFRVLSHRHRDGYNTADIEYLEDEKVEGPEFEELTALHESVYQQSVSWFASLQDHMKKQILSHFGSMPDREPEPQSNSSGPAWSWWILAVLPLERKAQLAILGMASLKERLLAIRRILVIITR

SEQ ID NO: 7 - RNA, 21 nt, synthetic construct
  source 1..21: mol_type = other RNA
ggagctggctcctgatgataa

SEQ ID NO: 8 - RNA, 21 nt, synthetic construct
  source 1..21: mol_type = other RNA
gctgcgggcggagttatattt

SEQ ID NO: 9 - RNA, 19 nt, synthetic construct
  source 1..19: mol_type = other RNA
cgtacgcggaatacttcga

SEQ ID NO: 10 - DNA, 15 bp, synthetic construct
  source 1..15: mol_type = other DNA
acgtgtgctgtccgt

SEQ ID NO: 11 - DNA, 21 bp, synthetic construct
  source 1..21: mol_type = other DNA
ggcgttagaaagcatccttcc

SEQ ID NO: 12 - DNA, 20 bp, synthetic construct
  source 1..20: mol_type = other DNA
gcagagggcacactcaaagt

SEQ ID NO: 13 - DNA, 20 bp, synthetic construct
  source 1..20: mol_type = other DNA
cccaacgcaccgaatagtta

SEQ ID NO: 14 - DNA, 18 bp, synthetic construct
  source 1..18: mol_type = other DNA
accagcgtgtccaggaag

SEQ ID NO: 15 - DNA, 21 bp, synthetic construct
  source 1..21: mol_type = other DNA
ggagcgagatccctccaaaat

SEQ ID NO: 16 - DNA, 23 bp, synthetic construct
  source 1..23: mol_type = other DNA
ggctgttgtcatacttctcatgg

SEQ ID NO: 17 - DNA, 22 bp, synthetic construct
  source 1..22: mol_type = other DNA
accaaggggcaccatataaaag

SEQ ID NO: 18 - DNA, 19 bp, synthetic construct
  source 1..19: mol_type = other DNA
cttcgccgagttgcactca

SEQ ID NO: 19 - DNA, 20 bp, synthetic construct
  source 1..20: mol_type = other DNA
cgcaggttcttggtcactgt

SEQ ID NO: 20 - DNA, 20 bp, synthetic construct
  source 1..20: mol_type = other DNA
tgttcacgaaagccagagcg

SEQ ID NO: 21 - DNA, 23 bp, synthetic construct
  source 1..23: mol_type = other DNA
catcactgccacccagaagactg

SEQ ID NO: 22 - DNA, 23 bp, synthetic construct
  source 1..23: mol_type = other DNA
atgccagtgagcttcccgttcag

SEQ ID NO: 23 - DNA, 20 bp, synthetic construct
  source 1..20: mol_type = other DNA
gaactatgatcaagccctgc

SEQ ID NO: 24 - DNA, 28 bp, synthetic construct
  source 1..28: mol_type = other DNA
gctaacttatgaataaaataccatgacc

SEQ ID NO: 25 - DNA, 34 bp, synthetic construct
  source 1..34: mol_type = other DNA
tggaaacaatgtattttaaaatgcagtcatttat

(19) United States
(12) Patent Application Publication
(10) Pub. No.: US 2026/0043817 A1
(43) Pub. Date: Feb. 12, 2026

(54) AGENT FOR DETECTING STRUCTURALLY ABNORMAL PROTEIN AND AGENT FOR REDUCING STRUCTURALLY ABNORMAL PROTEIN

(71) Applicant: The University of Tokyo, Tokyo (JP)

(72) Inventors: Makoto NAKANISHI, Tokyo (JP); Yoshikazu JOHMURA, Tokyo (JP)

(21) Appl. No.: 19/100,135

(22) PCT Filed: Aug. 1, 2023
(86) PCT No.: PCT/JP2023/028159

§ 371 Date: Jan. 31, 2025

(30) Foreign Application Priority Data
Aug. 1, 2022 (JP) 2022-122903

(51) Int. Cl. G01N 33/68; A01K 67/0276; C12N 9/02; C12Q 1/26; G01N 33/58

(52) U.S. Cl. CPC G01N 33/6896; A01K 67/0276; C12N 9/0069; C12Q 1/26; G01N 33/581; A01K 2267/0318; C12Y 113/12007; G01N 2333/90241; G01N 2800/28; G01N 2800/50

(57) ABSTRACT

The present invention provides a polypeptide that specifically recognizes structurally abnormal proteins generated in mammals due to misfolding or the like, and an agent for detecting or reducing structurally abnormal proteins that uses the polypeptide. The present invention relates to an agent for detecting structurally abnormal proteins, comprising as an active ingredient a polypeptide having structurally abnormal proteins-binding site which is a polypeptide consisting of the amino acid sequence represented by SEQ ID NO:2, or a polypeptide consisting of an amino acid sequence having 90% or more sequence identity with the amino acid sequence and having binding activity to structurally abnormal proteins, and an agent for reducing structurally abnormal proteins comprising as an active ingredient a polypeptide containing the structurally abnormal protein binding site and a ubiquitin ligase active site.

### TECHNICAL FIELD

The present invention relates to a polypeptide that specifically recognizes structurally abnormal proteins such as misfolded proteins, and to an agent for detecting or reducing structurally abnormal proteins using said polypeptide.

Priority is claimed on Japanese Patent Application No 2022-122903, filed in Japan on Aug. 1, 2022, the contents of which are incorporated herein by reference.

### BACKGROUND ART

Protein misfolding is a major cause of age-related frailty and disease. To avoid this, all cells have evolved protein quality control (PQC) systems that include molecular chaperone activity and protein degradation via the proteasome or autophagy (Non-Patent Literature 1-4). The PQC system varies depending on the cell type (postmitotic) and the subcellular compartment (cytoplasm, mitochondria, nucleus) (Non-Patent Literature 3, 5 and 6). For example, Lon is a member of the AAA+ superfamily of proteases that plays a pivotal role in bacterial and mitochondrial PQC by degrading damaged and misfolded proteins. The Lon substrate-binding (LonSB) domain (Pfam (https://pfam.xfam.org/) ID number: PF02190) is a conserved domain found in bacterial and mitochondrial PQC LON proteases and is responsible for binding to misfolded proteins (Non-Patent Literature 7-9). Mammals also have numerous PQC systems, but their specific mechanisms of action are largely unknown.

In post-mitotic cells, nuclear and cytosolic compartments never have a chance to intermingle. For this reason, nuclear PQC is particularly important in terminally differentiated neurons. In mammals, the nuclear SUMO-targeted ubiquitin system including PML and RNF4 has been proposed to function as nuclear PQC (Non-Patent Literature 10 and 11). However, a key characteristic of PQC ligase to destroy structurally abnormal proteins that share the same primary structure with their normal counterparts has not yet been characterized in the nuclear SUMO-targeted ubiquitin system. In addition, the sole knockout of PML in mice failed to reveal neurodegenerative phenotypes, suggesting the presence of other nuclear PQC ligases in mammals.

Cytoplasmic and intranuclear aggregations of TAR DNA-binding protein 43 (TDP-43) are common hallmarks of several neurodegenerative diseases such as amyotrophic lateral sclerosis (ALS) and frontotemporal lobar degeneration (FTLD) (Non-Patent Literature 12-14). TDP43 inclusions are also frequently detected in other neurodegenerative disorders such as Alzheimer's disease, Parkinson's disease, and Huntington's disease. TDP43 mainly localizes in the nucleus but is also shuttled to the cytoplasm where exerts various physiological functions. Cytoplasmic TDP43 inclusions contain TDP43 in its abnormally ubiquitylated and hyper-phosphorylated form (Non-Patent Literature 15 and 16), suggesting that the aberrant post-translational modifications of TDP43 have a role in the formation of inclusions. However, nuclear PQC ubiquitin ligases that selectively destroy structurally abnormal proteins have not yet been identified in mammals.

### CITATION LIST

### Patent Literatures

### [Patent Literature 1]

International Patent Publication No. 2020/095971

### [Non Patent Literatures]

### [Non Patent Literature 1]

Chen et al, Cold Spring Harbor Perspectives in Biology, 2011, vol. 3, p. 1-14.

### [Non Patent Literature 2]

Pilla et al, Annual Review of Cell and Developmental Biology, 2017, vol. 33, p. 439-465.

### [Non Patent Literature 3]

Amm et al, Biochimica et Biophysica Acta—Molecular Cell Research, 2014, vol. 1843, p. 182-1⁹⁶.

### [Non Patent Literature 4]

Wolff et al, Cell, 2014, vol. 157, p. 52-6⁴.

### [Non Patent Literature 5]

Fredrickson et al, Seminars in Cell and Developmental Biology, 2012, vol. 23(5), p. 530-537.

### [Non Patent Literature 6]

Joazeiro, Nature Reviews Molecular Cell Biology, 2019, vol. 20(6), p. 368-3⁸³.

### [Non Patent Literature 7]

Swamy et al, Nature, 1981, vol. 292, p. 652-6⁵⁴.

### [Non Patent Literature 8]

Wang et al, Proceedings of the National Academy of Sciences of the United States of America, 1993, vol. 90, p. 11247-1¹²⁵¹.

### [Non Patent Literature 9]

Amerik et al, FEBS letters, 1994, vol. 340, p. 25-2⁸.

### [Non Patent Literature 10]

Keiten-Schmitz et al, Molecular Cell, 2020, vol. 79, p. 54-67.e1-e7.

### [Non Patent Literature 11]

Guo et al, Molecular Cell, 2014, vol. 55, p. 15-3⁰.

### [Non Patent Literature 12]

Ling et al, Neuron, 2013, vol. 79(3), p. 416-4³⁸.

### [Non Patent Literature 13]

Prasad et al, Frontiers in Molecular Neuroscience, 2019, vol. 12, Article 25.

### [Non Patent Literature 14]

Wood et al, International Journal of Molecular Sciences, 2021, vol. 22, 4705.

### [Non Patent Literature 15]

Hasegawa et al, Annals of Neurology, 2008, vol. 64(1), p. 60-7⁰.

### [Non Patent Literature 16]

Neumann et al, Science, 2006, vol. 314, p. 130-1³³.

### [Non Patent Literature 17]

Johmura et al, Science, 2021, vol. 371, p. 265-2⁷⁰.

### [Non Patent Literature 18]

Johmura et al, Journal of Clinical Investigation, 2018, vol. 128(12), p. 5603-5619.

### [Non Patent Literature 19]

Nishiyama et al, Nature, 2013, vol. 502, p. 249-2⁵³.

### [Non Patent Literature 20]

Gur and Sauer, Genes & Development, 2008, vol. 22, p. 2267-2²⁷⁷.

### [Non Patent Literature 21]

Gupta et al, Nature Methods, 2011, vol. 8(10), p. 879-884.

### [Non Patent Literature 22]

Miyazaki et at, Nature Communications, 2016, 7:11689.

### [Non Patent Literature 23]

Kaneko et al, Scientific Reports, 2014, 4:6382.

### [Non Patent Literature 24]

Takeo and Nakagata, Biology of Reproduction, 2011, vol. 85, p. 1066-1⁰⁷².

### [Non Patent Literature 25]

Park et at, Experimental & Molecular Medicine, 2016, vol. 48, e276.

### [Non Patent Literature 26]

Niethamer et al, Stem Cell Reports, 2017, vol. 8, p. 529-5³⁷.

### [Non Patent Literature 27]

Miyoshi et al, EMBO Molecular Medicine, 2017, vol. 9, p. 880-889.

### SUMMARY OF INVENTION

### Technical Problem

The main object of the present invention is to provide a polypeptide that specifically recognizes structurally abnormal proteins generated by misfolding or the like in mammals, and an agent for detecting or reducing structurally abnormal proteins that utilizes said polypeptide.

### Solution to Problem

The inventors of the present invention have found that LONRF2 (LON peptidase N-terminal domain and RING finger protein 2), a member of the LONRF family of ubiquitin ligases, is a PQC ubiquitin ligase that binds to and ubiquitinates structurally abnormal proteins, and have completed the present invention.

The present invention includes the following agent for detecting structurally abnormal proteins and so on.

[1] An agent for detecting structurally abnormal proteins, comprising as an active ingredient a polypeptide having structurally abnormal proteins-binding site,
wherein the structurally abnormal protein-binding site is
   (A) a polypeptide consisting of the amino acid sequence represented by SEQ ID NO:2, or
   (B) a polypeptide consisting of an amino acid sequence having 90% or more sequence identity with the amino acid sequence represented by SEQ ID NO:2 and having binding activity to structurally abnormal proteins.
   [2] An agent for detecting structurally abnormal proteins, comprising as an active ingredient a functional nucleic acid for expressing a polypeptide having structurally abnormal proteins-binding site in host cells,
wherein the structurally abnormal protein-binding site is
(A) a polypeptide consisting of the amino acid sequence represented by SEQ ID NO:2, or
(B) a polypeptide consisting of an amino acid sequence having 90% or more sequence identity with the amino acid sequence represented by SEQ ID NO:2 and having binding activity to structurally abnormal proteins.
   [3] The agent for detecting structurally abnormal proteins according to [1] or [2],
wherein the polypeptide having binding activity to structurally abnormal proteins does not bind to a wild-type protein of firefly luciferase, but has binding activity to an R188Q/R261Q double mutant protein of firefly luciferase,
the wild-type protein of firefly luciferase consists of the amino acid sequence represented by SEQ ID NO:3, and
the mutant protein of firefly luciferase has the amino acid sequence represented by SEQ ID NO:4.
   [4] An agent for reducing structurally abnormal proteins, comprising as an active ingredient a polypeptide containing
(A1) a polypeptide consisting of the amino acid sequence represented by SEQ ID NO:1, or
(B1) a polypeptide consisting of an amino acid sequence having 90% or more sequence identity with the amino acid sequence represented by SEQ ID NO:1 and having binding activity to structurally abnormal proteins and ubiquitin ligase activity.
   [5] An agent for reducing structurally abnormal proteins, consisting of a polypeptide containing
(A1) a polypeptide consisting of the amino acid sequence represented by SEQ ID NO:1, or
(B1) a polypeptide consisting of an amino acid sequence having 90% or more sequence identity with the amino acid sequence represented by SEQ ID NO:1 and having binding activity to structurally abnormal proteins and ubiquitin ligase activity.
   [6] The agent for reducing structurally abnormal proteins according to [4] or [5],
wherein the polypeptide having binding activity to structurally abnormal proteins does not bind to a wild-type protein of firefly luciferase, but has binding activity to an R188Q/R261Q double mutant protein of firefly luciferase,
the wild-type protein of firefly luciferase consists of the amino acid sequence represented by SEQ ID NO:3, and
the mutant protein of firefly luciferase has the amino acid sequence represented by SEQ ID NO:4.
   [7]A pharmaceutical composition comprising, as an active ingredient, the agent for detecting structurally abnormal proteins according to any one of [1] to [3], or the agent for reducing structurally abnormal proteins according to [4] or [5].
   [8] The pharmaceutical composition according to [7], which is used for the treatment or prevention of a disease in which the structurally abnormal protein accumulates in a living body.
   [9] The pharmaceutical composition according to [8], wherein the disease is a neurodegenerative disease.
   [10] The pharmaceutical composition according to [9], wherein the neurodegenerative disease is amyotrophic lateral sclerosis.
   [11] The pharmaceutical composition according to [8], wherein the structurally abnormal protein is a misfolded protein.
   [12]A transgenic animal (excluding human) that has a deletion of the LONRF2 gene or has a mutation that reduces its function, and is used as a model for an amyotrophic lateral sclerosis.
   [13] The transgenic animal according to [12], wherein the mutation is a V599M mutation.
   [14]A cell obtained from the transgenic animal according to [12] or [13].
   [15]A method for evaluating the risk of developing a disease, comprising:
a typing step of typing the genotype of rs143848902 of a human subject; and
an evaluation step of evaluating the subject's risk of developing a disease in which abnormal proteins accumulate in the body based on the typing results obtained by the typing step, and
wherein the subject is evaluated as having a high risk of developing the disease when the genotype of rs143848902 of the subject is the ATG type.
   [16] The method for evaluating the risk of developing a disease according to [15], wherein the disease is amyotrophic lateral sclerosis.
   [17]A pharmaceutical composition comprising, a functional nucleic acid for expressing the human LONRF2 gene as an active ingredient, and
wherein it is for use in treating or preventing amyotrophic lateral sclerosis.

### Advantageous Effects of Invention

The agent for detecting structurally abnormal proteins according to the present invention contains as an active ingredient a polypeptide that specifically binds to structurally abnormal proteins generated in mammalian cells through misfolding and so on. Therefore, the agent for detecting structurally abnormal proteins, an agent for reducing structurally abnormal proteins using the polypeptide, and pharmaceutical compositions containing the agents as an active ingredient are useful for preventing and treating various diseases caused by the accumulation of structurally abnormal proteins, improving functional decline due to aging and so on.

The transgenic animal according to the present invention has a deletion or reduced function of the LONRF2 gene. The transgenic animal therefore accumulates structurally abnormal proteins particularly in the nervous system and is useful as an ALS model.

### BRIEF DESCRIPTION OF DRAWINGS

FIG. 1 shows the relative expression levels of LONRF2 and p16 obtained by qPCR analysis using RNA from primary cerebral cortical neuron cells cultured for 1 day (P1) or 14 days (P14) in Example 1.

FIG. 2 shows the results of measuring the fluorescence intensity of d-Sen cells expressing FLAG-LONRF2 by doxycycline (Dox) induction in the presence or absence of Dox (1 mg/mL) by proteostat staining in Example 1.

FIG. 3 shows the results of measuring the fluorescence intensity of d-Sen cells expressing shRNA (shLONRF2-1, shLONRF2-2, or shControl) by Dox induction when were cultured in the presence of Dox (1 mg/mL) by proteostat staining in Example 1.

FIG. 4 shows the results of immunoblotting using an anti-HA antibody on cell lysates of HeLa cells that were cultured in the presence of CHX after coexpressing FLAG-LONRF2-WT or mock with HA-tagged wild-type luciferase (Fluc-HA-WT) or HA-tagged Fluc-DM (Fluc-HA-DM) in Example 1.

FIG. 5 shows the results of measuring luciferase activity of cell lysates of HeLa cells that were cultured in the presence of CHX after co-expressing FLAG-LONRF2-WT, FLAG-LONRF2-ATPR, FLAG-LONRF2-ARING1, FLAG-LONRF2-ARING2, or FLAG-LONRF2-ALonSB with Fluc-HA-DM in Example 1.

FIG. 6 shows the results of an in vivo ubiquitination assay performed on HeLa cells co-expressing LONRF2-WT, FLAG-Fluc-WT or FLAG-Fluc-DM with HA-Ub in Example 1.

FIG. 7 shows the results of immunoprecipitation and immunoblotting on cell lysates of HeLa cells that were cultured for 48 hours after co-expressing FLAG-LONRF2-WT, FLAG-LONRF2-RINGm (C4A), or FLAG-LONRF2-LonSBm (P5A) with Fluc-HA-WT or Fluc-HA-DM in Example 1.

FIG. 8 shows the results of immunoblotting using anti-HA antibody on cell lysates of HeLa cells that were cultured in the presence of CHX or in the presence of CHX and Shield-1 after co-expressing FLAG-LONRF2-WT or mock with NLS-AgDD-HA in Example 1.

FIG. 9 shows the results of an in vivo ubiquitination assay performed on HeLa cells co-expressing LONRF2-WT, FLAG-AgDD, and HA-Ub in the presence or absence of Shield-lin Example 1.

FIG. 10 shows the results of immunoprecipitation and immunoblotting on cell lysates of HeLa cells that were cultured for 48 hours in the presence or absence of Shield-1 after co-expressing FLAG-LONRF2-WT, FLAG-LONRF2-RINGm (C4A), or FLAG-LONRF2-LonSBm (P5A) with NLS-AgDD-HA in Example 1.

FIG. 11 shows the results of measuring the rate (%) of stress granule-positive cells, in the total cells (n=200), which were transfected with Dox-inducible shLONRF2-1, Dox-inducible shLONRF2-2, or Dox-inducible shControl, cultured in the presence of Dox (1 mg/mL) and then treated with sodium arsenite and washed out in Example 1.

FIG. 12 shows the results of an in vivo ubiquitination assay on A549 cells co-expressing FLAG-hnRNP M1 and HA-Ub with LONRF2-WT, FLAG-LONRF2-RINGm (C4A), or FLAG-LONRF2-LonSBm (P5A) in the presence or absence of sodium arsenite in Example 2.

FIG. 13 shows the results of an in vivo ubiquitination assay on A549 cells co-expressing FLAG-TDP43 and HA-Ub with LONRF2-WT, FLAG-LONRF2-RJNGm (C4A), or FLAG-LONRF2-LonSBm (P5A) in the presence or absence of sodium arsenite in Example 2.

FIG. 14 shows the results of immunoprecipitation and immunoblotting on cell lysates of A549 cells that were cultured for 30 minutes in the presence or absence of sodium arsenite after co-expressing FLAG-LONRF2-WT, FLAG-LONRF2-RINGm (C4A), or FLAG-LONRF2-LonSBm (P5A) in Example 2.

FIG. 15 shows the results of immunoprecipitation and immunoblotting on cell lysates of A549 cells co-expressing Dox-inducible shLONRF2-1 with LONRF2-WT, LONRF2-RINGm (C4A) or LONRF2-LonSBm (P5A) and cultured in the presence of Dox and then in the presence of sodium arsenite in Example 2.

FIG. 16 shows the alignment of nucleotide sequences of a partial region of exon 2 of the LONRF2 gene in a LONRF2-WT mouse (LONRF2^{+/+} ) and a LONRF2-KO mouse (LONRF2^{-/-}) generated in Example 3.

FIG. 17 shows the results of investigating the motor functions of 3-month-old and 21-month-old LONRF2-WT and LONRF2-KO mice in Example 3.

FIG. 18 shows the results of measuring the number of ChAT-positive motor neurons near anterior horn in the lumbar spinal cord (A), the number of NeuN-positive neurons per square millimeter in the lumbar spinal cord (B), and the number of Fluoro Jade C-positive degenerated neurons per square millimeter in the lumbar spinal cord (C) of 21-month-old LONRF2-WT and LONRF2-KO mice in Example 3.

FIG. 19 shows the results of measuring the rate (%) of Ataxin2-positive inclusion-containing cells per square millimeter in the lumbar spinal cord (A), the rate (%) of G3BP1-positive inclusion-containing cells (B), and the rate (%) of phospho-TDP43-positive inclusion-containing cells (C) of 21-month-old LONRF2-WT and LONRF2-KO mice in Example 3.

FIG. 20 shows an outline of the culture protocol of differentiating iPS cells prepared from mouse fibroblasts into motor neurons in Example 3.

FIG. 21 shows the results of measuring the relative amount of LONRF2 mRNA in cells at each differentiation stage during differentiation of LONRF2^{+/+} iPS cells into motor neurons in Example 3.

FIG. 22 shows the results of measuring the relative amount of LONRF2 mRNA (A) and the relative amount of p16 mRNA (B) in motor neurons differentiated from LONRF2^{+/+} iPS cells before culture (day 0) and after 14 days of culture in Example 3.

FIG. 23 shows the results of measuring the length (μm) of neurites before and after culture of motor neurons differentiated from LONRF2^{+/+} iPS cells and of motor neurons differentiated from LONRF2^{-/-}iPS cells in Example 3.

FIG. 24 shows the results of measuring the survival rate (%) before and after culture of motor neurons differentiated from LONRF2^{+/+} iPS cells and of motor neurons differentiated from LONRF2^{-/-}iPS cells in Example 3.

FIG. 25 shows the results of measuring the rate (%) of pTDP43 positive cells before and after culture of motor neurons differentiated from LONRF2^{+/+} iPS cells and of motor neurons differentiated from LONRF2^{-/-}iPS cells in Example 3.

FIG. 26 shows the results of measuring the rate (%) of G3BP1 positive cells before and after culture of motor neurons differentiated from LONRF2^{+/+} iPS cells and of motor neurons differentiated from LONRF2^{-/-}iPS cells in Example 3.

FIG. 27 shows the results of an in vivo ubiquitination assay on HeLa cells co-expressing FLAG-TDP43 and HA-Ub with LONRF2-WT or various single amino acid mutants of LONRF2 in the presence of sodium arsenite in Example 4.

FIG. 28 shows the results of immunoprecipitation and immunoblotting on cell lysates of A549 cells co-expressing LONRF2-WT or various single amino acid mutants of LONRF2 and cultured in the presence of sodium arsenite for 30 minutes in Example 4.

FIG. 29 shows the results of measuring the rate (%) of stress granule-positive cells, in the total cells (n=200), which were transfected with Dox-inducible shLONRF2-1 or Dox-inducible shControl cultured in the presence of Dox (1 mg/mL) and then treated with sodium arsenite and washed out in Example 4.

FIG. 30 shows the results of immunoprecipitation and immunoblotting on cell lysates of A549 cells co-expressing Dox-inducible shLONRF2-1 or Dox-inducible shControl with LONRF2-WT or LONRF2-V599M and cultured in the presence of Dox and then in the presence of sodium arsenite, and on cell lysate of A549 cells having been further washed in Example 4.

FIG. 31 shows the results of measuring the forelimb grip strength (g) (FIG. 31(A)), the forelimb+hindlimb grip strength (g) (FIG. 31(B)), and the rotor rod test (seconds) (FIG. 31(C)), of an ALS mouse model group administered with AAV-FLAG-LONRF2 (AAv group) and an ALS mouse model group administered with AAV-EGFP (Control group) in Example 5.

### DESCRIPTION OF EMBODIMENTS

In order for a protein to perform its inherent function, the amino acid chain, which is its primary structure, must be correctly folded into a specific three-dimensional structure. In the present invention and this specification, when a protein has a three-dimensional structure that allows it to perform its inherent function, the structure of the protein is said to be normal, and such a protein is called a “normal protein.”

In the present invention and this specification, a “structurally abnormal protein” is a protein whose three-dimensional structure has changed from its original structure, and whose function and/or properties have changed. In the present invention and this specification, a structural change that changes the original function or characteristics of a protein may be referred to as a structural abnormality. A protein having its original structure may be referred to as a normal protein. Structurally abnormal proteins often cause diseases by exhibiting reduced or lost activity or function, or toxicity, as compared with normal proteins. Structurally abnormal proteins include proteins that have a normal or nearly normal folding structure (higher-order structure) but whose activity or function have reduced or lost compared to normal proteins, misfolded proteins that have an abnormal folding structure but whose activity or function remains, and misfolded proteins that have an abnormal folding structure and whose activity or function have reduced or lost.

Causes of structural abnormalities include, for example, mutations in constituent amino acids, abnormalities in post-translational modifications, abnormalities in chaperones (proteins that help fold proteins), and environmental factors such as oxidative stress and ER stress. The agent for detecting structurally abnormal proteins relate to the present invention can detect a large number of structural abnormality proteins with different causes of structural abnormality. As the target structural abnormality protein detected by the agent for detecting structurally abnormal proteins relate to the present invention, structural abnormality proteins that are causes or markers of various diseases such as cancer and neurodegenerative diseases are preferred.

In the present invention and this specification, a polypeptide that does not bind to a normal protein but can bind to a conformationally abnormal protein is referred to as a “structurally abnormal protein-specific binding domain.”

The ubiquitin ligase LONRF (LON peptidase N-terminal domain and RING finger protein) family, which contains a RING finger domain and a LonSB domain, has three isozymes (LONRF1 to 3) in mammals. LONRF2 has, from the N-terminus, a TPR domain, two RING finger domains, and a LonSB domain. The TPR domain is a domain that interacts with proteins. In the present specification, of the two RING finger domains, the N-terminal one is called RING finger domain 1 and the C-terminal one is called RING finger domain 2.

As shown in the Examples below, the present inventors have found for the first time that LONRF2 is a mammalian PQC ubiquitin ligase, and that the LonSB domain in LONRF2 is a polypeptide that does not bind to normal proteins but binds to structurally abnormal proteins. In other words, the LonSB domain in LONRF2 is structurally abnormal proteins-specific binding domain.

Generally, a protein having some physiological activity may have one or more amino acids deleted, substituted, or added without impairing the physiological activity. That is, the LonSB domain of LONRF2 can also have one or more amino acids deleted, substituted or added without losing its binding activity to structurally abnormal proteins.

In the present invention and this specification, “an amino acid is deleted from a polypeptide” means that an amino acid constituting the polypeptide is lost (removed).

In the present invention and this specification, “an amino acid is substituted in a polypeptide” means that an amino acid constituting the polypeptide is changed to another amino acid.

In the present invention and this specification, “an amino acid is added in a polypeptide” means that a new amino acid is inserted into the polypeptide.

### <Agent for Detecting Structurally Abnormal Proteins>

The agent for detecting structurally abnormal proteins relate to the present invention contains, as an active ingredient, a polypeptide that contains a conformational abnormal protein-specific binding domain as structurally abnormal proteins-binding site. The structurally abnormal protein-binding domain in the agent for detecting structurally abnormal proteins relate to the present invention is the LonSB domain (a region from amino acid 538 to amino acid 738 in the amino acid sequence of NP_940863.3) (SEQ ID NO: 2) of human LONRF2 (NCBI Reference Sequence: NP_940863.3) (SEQ ID NO: 1, 754 aa) or a mutant thereof. Specifically, the agent for detecting structurally abnormal proteins relate to the present invention contains as an active ingredient a polypeptide containing structurally abnormal proteins-specific binding domain consisting of the following (A) or (B):

(A) a polypeptide consisting of the amino acid sequence represented by SEQ ID NO:2, or
(B) a polypeptide consisting of an amino acid sequence having 90% or more sequence identity with the amino acid sequence represented by SEQ ID NO:2 and having binding activity to structurally abnormal proteins.

TABLE 1

| | | Seq. |
|---|---|---|
| | Sequence | No. |
| human | MSPEPVPPPPPPQCPGCDRAEPIAQRLEEGDEAFRAGDYEMA | 1 |
| LONRF2 | AELFRSMLAGLAQPDRGLCLRLGDALARAGRLPEALGAFRGA | |
| | ARLGALRPEELEELAGGLVRAVGLRDRPLSAENPGGEPEAPG | |
| | EGGPAPEPRAPRDLLGCPRCRRLLHKPVTLPCGLTVCKRCVE | |
| | PGPARPQVRRVNVVLSGLLEKCFPAECRLRRLAGQARSLQRQ | |
| | QQPEAALLRCDQALELAPDDNSLLLLRAELYLTMKNYEQALQ | |
| | DASAACQNEPLLIKGHQVKAQALSGLGRSKEVLKEFLYCLAL | |
| | NPECNSVKKEAQKVMCEVLFSATANVHENLTSSIQSRLKAQG | |
| | HSHMNAQALLEEGDAGSSENSSEKSDMLGNINSSVLYFILGL | |
| | HFEEDKKALESILPTAPSAGLKRQFPDDVEDAPDLNAPGKIP | |
| | KKDLSLQRSPNSETEESQGLSLDVIDFECALCMRLLFEPVTT | |
| | PCGHTFCLKCLERCLDHAPHCPLCKDKLSELLASRNFNITVL | |
| | AEELIFRYLPDELSDRKRIYDEEMSELSNLTRDVPIFVCAMA | |
| | FPTVPCPLHVFEPRYRLMIRRCMETGTKRFGMCLSAEHAGLS | |
| | EYGCMLEIKDVRTFPDGSSVVDAIGISRFRVLSHRHRDGYNT | |
| | ADIEYLEDEKVEGPEYEELAALHDSVHQQSVSWFASLQDRMK | |
| | EQILSHFGVMPDREPEPQSNPSGPAWSWWILAVLPLERKAQL | |
| | AILGMTSLKERLLAIRRILVIITRKMNSRQELANARERNN | |
| mouse | MSPEPAPPEPQGVRRSLEEPPEEPQGQGSAAFLAGDYEVAAE | |
| LONRF2 | LERSLLAGLAQPERGLCLQLGEALARAGRLPEAIGAFRGAAL | |
| | LGPLQPGELSELASGLARALGLREKRPPVAARPSGCASGAPG | |
| | PVAPRDLLGCPRCGRLLYKPVILSSGLTVCKRCVEQVPVRAQ | |
| | ARRVNVMLSGLLERCFPAECRLRRLACQARGLHRQQQPEAAL | 5 |
| | LRCQQALDMAPDDNSLLLLRAELYLTMKNYDQALQDAEAVCQ | |
| | REPLLTKGHHIKAQILSGLGRHREVLKEFIYCLALNPECNSA | |
| | KKETQKVICEVFFSTSESEHQTSTSSTETGPEALCEGQTNPQ | |
| | YPLEEAGGHANADNPKTPSEKSDAPADINSSVLYFILGLHCE | |
| | EDKKALEGIVPAAPSSTLKRQLPSDAQDDEELKANTPEKIPK | |
| | KDADSPPQRNASSLEEEPEFTIDATDFECALCMRLLFEPVIT | |
| | PCGHTFCLKCLERCLDHAPHCPLCKDKLSELLATRNFNVTVL | |
| | TEELIFRYLPDELSDRKRVYDEEMSELSNLTRDVPIFVCAMA | |
| | FPTVPCPLHVFEPRYRLMIRRCMETGTKRFGMCLSAENAGIS | |
| | EYGCMLEIKDVRTFPDGSSVVDAIGISRFRVLSHRHRDGYNT | |
| | ADIEYLEDEKVEGPEFEELTALHESVYQQSVSWFASLQDHMK | |
| | KQILSHFGSMPDREPEPQSNSSGPAWSWWILAVLPLERKAQL | |
| | AILGMASLKERLLAIRRILVIITRKLNSRQEMANNTQRDN | |

The amino acid sequence of human LONRF2 is shown in Table 1. In the table, the underlined portion is the LonSB domain.

Hereinafter, the structurally abnormal protein-specific binding domain consisting of the polypeptide (A) may be referred to as a hLONRF2-LonSB domain, and the structurally abnormal protein-specific binding domain consisting of the polypeptide (B) may be referred to as a hLONRF2-LonSBm domain.

The sequence identity (homology) between amino acid sequences is determined by aligning two amino acid sequences with gaps at the insertion and deletion sites so that the corresponding amino acids are most identical, and calculating the ratio of identical amino acids to the entire amino acid sequence excluding gaps in the alignment obtained. The sequence identity between amino acid sequences can be determined using various homology search software known in the art. The value of sequence identity between amino acid sequences in the present invention and this specification is obtained by calculation based on the alignment obtained by the known homology search software BLASTP.

In the polypeptide (B), the sequence identity with the amino acid sequence represented by SEQ ID NO:2 is not particularly limited as long as it is 90% or more but less than 100%. It is preferably 95% or more but less than 100%, and more preferably 98% or more but less than 100%.

The structurally abnormal protein-specific binding domain contained in the agent for detecting structurally abnormal proteins relate to the present invention may be a polypeptide having less than 90% sequence identity with the amino acid sequence represented by SEQ ID NO: 2, so long as it retains binding activity to structurally abnormal proteins. For example, the structurally abnormal protein-specific binding domain may be a polypeptide having sequence identity of 60% or more and less than 100%, preferably 70% or more and less than 100%, more preferably 80% or more and less than 100%, with the amino acid sequence represented by SEQ ID NO: 2, and having binding activity to structurally abnormal proteins.

Examples of the polypeptide (B) include the LonSB domain of a V538I mutant of human LONRF2 (a polypeptide in which the first amino acid in SEQ ID NO:2 is substituted from valine to isoleucine), the LonSB domain of an A585V mutant of human LONRF2 (a polypeptide in which the 48^{th} amino acid in SEQ ID NO:2 is substituted from alanine to valine), the LonSB domain of an A655V mutant of human LONRF2 (a polypeptide in which the 118^{th} amino acid in SEQ ID NO:2 is substituted from alanine to valine), the LonSB domain of a V705M mutant of human LONRF2 (a polypeptide in which the 168^{th} amino acid in SEQ ID NO:2 is substituted from valine to methionine), and the LonSB domain of an S721L mutant of human LONRF2 (a polypeptide in which the 184^{th} amino acid in SEQ ID NO:2 is substituted from serine to leucine).

Whether or not the polypeptide (B) retains binding activity to structurally abnormal proteins can be determined by using as an index the binding ability to a wild-type protein of firefly luciferase (a polypeptide consisting of the amino acid sequence represented by SEQ ID NO: 3; hereinafter, sometimes referred to as “wild-type luciferase”) and an R188Q/R261Q double mutant protein of firefly luciferase (a polypeptide consisting of the amino acid sequence represented by SEQ ID NO: 4; hereinafter, sometimes referred to as “aggregated mutant luciferase”). In the present invention and the present specification, a “polypeptide having binding activity to structurally abnormal proteins” is a polypeptide that does not bind to wild-type luciferase but has binding activity to an aggregated mutant luciferase.

TABLE 2

| | | Seq. |
|---|---|---|
| | Sequence | No. |
| Wild-type | MEDAKNIKKGPAPFYPLEDGTAGEQLHKAMKRYALVPGTIA | 3 |
| Luciferase | | |
| | FTDAHIEVNITYAEYFEMSVRLAEAMKRYGLNTNHRIVVCS | |
| | ENSLOFFMPVLGALFIGVAVAPANDIYNERELLNSMNISQP | |
| | TVVFVSKKGLQKILNVQKKLPIIQKIIIMDSKTDYQGFQSM | |
| | 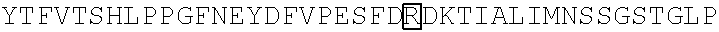 | |
| | KGVALPHRTACVRFSHARDPIFGNQIIPDTAILSVVPFHHG | |
| | 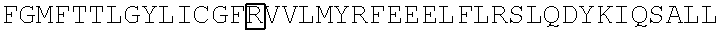 | |
| | VPTLFSFFAKSTLIDKYDLSNLHEIASGGAPLSKEVGEAVA | |
| | KRFHLPGIRQGYGLTETTSAILITPEGDDKPGAVGKVVPFF | |
| | EAKVVDLDTGKTLGVNQRGELCVRGPMIMSGYVNNPEATNA | |
| | LIDKDGWLHSGDIAYWDEDEHFFIVDRLKSLIKYKGYQVAP | |
| | AELESILLQHPNIFDAGVAGLPDDDAGELPAAVVVLEHGKT | |
| | MTEKEIVDYVASQVTTAK KLRGGVVFVDEVPKGLTGKLDA | |
| | RKIREILIKAKKGGKSKL | |
| Aggregated mutant | MEDAKNIKKGPAPFYPLEDGTAGEQLHKAMKRYALVPGTIA | 4 |
| Luciferase | | |
| | FTDAHIEVNITYAEYFEMSVRLAEAMKRYGLNINHRIVVCS | |
| | ENSLQFFMPVLGALFIGVAVAPANDIYNERELLNSMNISQP | |
| | TVVFVSKKGLQKILNVQKKLPIIQKIIIMDSKTDYQGFQSM | |
| | 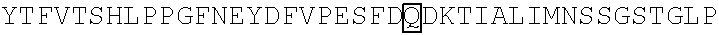 | |
| | KGVALPHRTACVRFSHARDPIFGNQIIPDTAILSVVPFHHG | |
| | 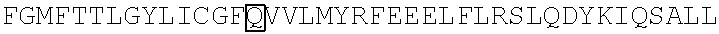 | |
| | VPTLFSFFAKSTLIDKYDLSNLHEIASGGAPLSKEVGEAVA | |
| | KRFHLPGIRQGYGLTETTSAILITPEGDDKPGAVGKVVPFF | |
| | EAKVVDLDTGKTLGVNQRGELCVRGPMIMSGYVNNPEATNA | |
| | LIDKDGWLHSGDIAYWDEDEHFFIVDRLKSLIKYKGYQVAP | |
| | AELESILLQHPNIFDAGVAGLPDDDAGELPAAVVVLEHGKT | |
| | MTEKEIVDYVASQVTTAKKLRGGVVFVDEVPKGLIGKLDAR | |
| | KIREILIKAKKGGKSKL | |

The binding activity to the aggregated mutant luciferase can be measured by various methods capable of measuring the interaction between two types of proteins. Examples of the measurement methods include the co-immunoprecipitation method, the pull-down assay method, the far-western blotting method, the surface plasmon resonance method, and the FRET (Fluorescence resonance energy transfer) method. These can be performed by standard methods.

The hLONRF2-LonSB domain and the hLONRF2-LonSBm domain can bind to various structurally abnormal proteins in addition to the aggregated mutant luciferase. Examples of structurally abnormal proteins to which the hLONRF2-LonSB domain and the hLONRF2-LonSBm domain bind include misfolded proteins such as TDP43, α-synuclein, polyglutamine, tau, amyloid P, prion, P2-microglobulin, transthyretin, and immunoglobulin L chain. These misfolded proteins are structurally abnormal proteins that are the cause or marker of neurodegenerative diseases. In addition, the hLONRF2-LonSB domain and the hLONRF2-LonSBm domain can also bind to and detect structurally abnormal proteins due to amino acid mutations, such as loss-of-function mutant p53 proteins.

The polypeptide (B) may be an artificially designed one, or may be the LonSB domain of a homologue of human LONRF2 or a mutant thereof. For example, the LonSB domain (mLONRF2-LonSB domain) (SEQ ID NO: 6, the underlined portion in SEQ ID NO: 5 in Table 1) of mouse LONRF2 (SEQ ID NO: 5) also binds to structurally abnormal proteins, similar to the hLONRF2-LonSB domain.

The polypeptide of the active ingredient of the agent for detecting structurally abnormal proteins relate to the present invention may be a polypeptide consisting of only structurally abnormal proteins specific binding domain, and may have a tag peptide, a labeling protein, a signal peptide, or the like at the N-terminus or C-terminus of the structurally abnormal protein specific binding domain. The tag peptide can be used a tag commonly used in the expression or purification of recombinant proteins, for example, a such as His tag, HA (hemagglutinin) tag, Myc tag, and FLAG tag. The labeling protein can be used a fluorescent protein or a protein that serves as a substrate or enzyme for chemiluminescence. The signal peptide can be used, for example, a nuclear localization signal (NLS) peptide, an endoplasmic reticulum retention signal peptide, and a secretory signal peptide.

The polypeptide of the active ingredient of the agent for detecting structurally abnormal proteins relate to the present invention may be a molecule bound to a component other than the polypeptide. For example, the agent for detecting structurally abnormal proteins relate to the present invention may be a molecule in which a polypeptide containing structurally abnormal proteins specific binding domain bound to sugars, nucleic acids, lipids, low molecular weight compounds, polymers such as polyethylene glycol and so on.

The polypeptides (A) and (B) may be chemically synthesized based on the amino acid sequence, or may be produced by a protein expression system using the polynucleotide relate to the present invention described below. The polypeptide (B) may also be artificially synthesized based on the polypeptide consisting of the amino acid sequence represented by SEQ ID NO:2 using gene recombination technology to introduce amino acid mutations.

The polypeptide of the active ingredient of the agent for detecting structurally abnormal proteins relate to the present invention may be a polypeptide consisting only of natural amino acids, a polypeptide containing modified amino acids, or a polypeptide containing corresponding artificial amino acids. The modifications include phosphorylation, glycosylation, nitrosylation, methylation, acetylation, sugar chain addition, lipid addition and so on. As the artificial amino acid, known artificial amino acids such as p-benzoylphenylalanine, 4-azidophenylalanine, 3-iodotyrosine, nitrotyrosine, tyrosine sulfate, azido-Z-lysine, acetyl-lysine and so on. can be used.

The polypeptide of the active ingredient of the agent for detecting structurally abnormal proteins relate to the present invention may be a functional nucleic acid for expressing a polypeptide containing structurally abnormal proteins specific binding domain in a host cell. The functional nucleic acid is not particularly limited as long as it can synthesize a polypeptide containing structurally abnormal proteins specific binding domain in a cell into which the functional nucleic acid is introduced. The functional nucleic acid may be DNA, RNA, or a chimeric nucleic acid containing DNA and RNA. In addition, it may be a nucleic acid consisting only of natural nucleotides, a nucleic acid containing modified nucleotides, or a nucleic acid containing an artificial nucleic acid. The modification may be methylation, methoxylation, pseudouridylation, deamination, thiolation and so on. The artificial nucleic acid may be BNA (Bridged Nucleic Acid), alkynyl nucleic acid and so on.

The functional nucleic acid may be, for example, a nucleic acid in which a polynucleotide containing a base sequence encoding a polypeptide containing the structurally abnormal protein-binding site is inserted into an expression vector. The expression vector may be a DNA vector, an RNA vector, or a viral vector. The expression vector may be appropriately selected from widely used expression vectors. The functional nucleic acid may be a linear nucleic acid or a circular nucleic acid.

The agent for detecting structurally abnormal proteins relate to the present invention specifically binds to structurally abnormal proteins because its active ingredient is a polypeptide including the hLONRF2-LonSB domain or the hLONRF2-LonSBm domain. By utilizing the specific binding ability to structurally abnormal proteins via the hLONRF2-LonSB domain or the hLONRF2-LonSBm domain, the agent for detecting structurally abnormal proteins relate to the present invention can detect structurally abnormal proteins while distinguishing it from normal proteins.

For example, when the active ingredient of the agent for detecting structurally abnormal proteins relate to the present invention is a polypeptide containing a hLONRF2-LonSB domain or a hLONRF2-LonSBm domain, the polypeptide can be used as the solid phase of an affinity column or an immune-strip to detect or purify structurally abnormal proteins from biological samples such as cell lysates and serum. When the active ingredient of the agent for detecting structurally abnormal proteins relate to the present invention is a functional nucleic acid for expressing a polypeptide containing a hLONRF2-LonSB domain or a hLONRF2-LonSBm domain and having an appropriate tag attached thereto in a cell, the functional nucleic acid can be introduced into a cell for expression, and then immunostaining can be performed using an antibody against the tag to detect structurally abnormal proteins such as aggregates in the cell.

### <Agent for Reducing Structurally Abnormal Proteins>

As shown in the Examples below, LONRF2 binds in cells to structurally abnormal proteins via the LonSB domain and ubiquitinates the structurally abnormal proteins via the RING finger domain, leading to degradation of the structurally abnormal proteins by intracellular enzymes. In other words, LONRF2 can function as an agent for reducing structurally abnormal proteins.

Specifically, the agent for reducing structurally abnormal proteins contains as an active ingredient a polypeptide comprising a polypeptide consisting of the following (A1) or (B1):
(A1) a polypeptide consisting of the amino acid sequence represented by SEQ ID NO:1, or
(B1) a polypeptide consisting of an amino acid sequence having 90% or more sequence identity with the amino acid sequence represented by SEQ ID NO:1 and having binding activity to structurally abnormal proteins and ubiquitin ligase activity.

The polypeptide (A1) is a full-length human LONRF2 protein (hLONRF2). Hereinafter, the polypeptide (B1) may be referred to as a hLONRF2 mutant.

In the polypeptide (B1), the sequence identity with the amino acid sequence represented by SEQ ID NO:2 is not particularly limited as long as it is 90% or more but less than 100%. Is is preferably 95% or more but less than 100%, and more preferably 98% or more but less than 100%.

The polypeptide of the active ingredient of the agent for reducing structurally abnormal proteins relate to the present invention may be a polypeptide having less than 90% sequence identity with the amino acid sequence represented by SEQ ID NO: 1, as long as it retains the activity of reducing structurally abnormal proteins. For example, the polypeptide of the active ingredient may be a polypeptide that has sequence identity of 60% or more but less than 100%, preferably 70% or more but less than 100%, and more preferably 80% or more but less than 100% with the amino acid sequence represented by SEQ ID NO: 1, and that has binding activity to a structurally abnormal protein and ubiquitin ligase activity.

Examples of hLONRF2 mutants include a V538I mutant of human LONRF2, an A585V mutant of human LONRF2, an A655V mutant of human LONRF2, a V705M mutant of human LONRF2, and an S721L mutant of human LONRF2.

The polypeptide (B1) may be an artificially designed one, and may be the LonSB domain of a homologue of human LONRF2 or a mutant thereof.

The polypeptide of the active ingredient of the agent for reducing structurally abnormal proteins relate to the present invention may be a polypeptide consisting only of hLONRF2 or a hLONRF2 mutant, and may have a tag peptide, a labeling protein, a signal peptide, or the like at its N-terminus or C-terminus. As the tag peptide, labeling protein, and signal peptide, those listed above can be used.

The polypeptide of the active ingredient of the agent for reducing structurally abnormal proteins relate to the present invention may be a molecule bound to a component other than the polypeptide. For example, the agent for reducing structurally abnormal proteins relate to the present invention may be a molecule in which a polypeptide containing hLONRF2 or an hLONRF2 mutant is bound to sugars, nucleic acids, lipids, low molecular weight compounds, polymers such as polyethylene glycol and so on.

The polypeptides (A1) and (B1) may be chemically synthesized based on the amino acid sequence, or may be produced by a protein expression system using the polynucleotide relate to the present invention described below. The polypeptide (B1) may also be artificially synthesized based on the polypeptide consisting of the amino acid sequence represented by SEQ ID NO:1 using gene recombination technology to introduce amino acid mutations.

The polypeptide of the active ingredient of the agent for reducing structurally abnormal proteins relate to the present invention may be a polypeptide consisting only of natural amino acids, a polypeptide containing modified amino acids, or a polypeptide containing corresponding artificial amino acids. As the modified and artificial amino acids, those listed above can be used.

The polypeptide of the agent for reducing structurally abnormal proteins relate to the present invention may be a functional nucleic acid for expressing a polypeptide containing hLONRF2 or an hLONRF2 mutant in a host cell. The functional nucleic acid is not particularly limited as long as it can synthesize a polypeptide containing hLONRF2 or an hLONRF2 mutant in a cell into which the functional nucleic acid is introduced. The functional nucleic acid may be DNA, RNA, or a chimeric nucleic acid containing DNA and RNA. In addition, it may be a nucleic acid consisting only of natural nucleotides, a nucleic acid containing modified nucleotides, or a nucleic acid containing an artificial nucleic acid. As the modified and artificial nucleotides, those listed above can be used.

The functional nucleic acid may be, for example, a nucleic acid in which a polynucleotide containing a base sequence encoding a polypeptide containing hLONRF2 or an hLONRF2 mutant is inserted into an expression vector. The expression vector may be a DNA vector, an RNA vector, or a viral vector. The expression vector may be appropriately selected from widely used expression vectors. The functional nucleic acid may be a linear nucleic acid or a circular nucleic acid. When used as an active ingredient in a pharmaceutical composition, adenovirus vectors are particularly preferred because of their proven effectiveness in gene therapy and the like.

hLONRF2 or hLONRF2 mutants can bind to and ubiquitinate various structurally abnormal proteins in addition to aggregated mutant luciferase. Therefore, the agent for reducing structurally abnormal proteins relate to the present invention is useful for reducing the amount of various structurally abnormal proteins. Examples of structurally abnormal proteins that can be reduced include misfolded proteins that are listed as proteins to which the hLONRF2-LonSB domain and the like can bind.

In particular, hLONRF2 is highly expressed in the nervous system of the brain, and therefore an agent for reducing structurally abnormal proteins, which contains a polypeptide containing hLONRF2 or an hLONRF2 mutant as an active ingredient, is particularly suitable for reducing misfolded proteins in nervous tissues.

### <Pharmaceutical Composition>

The agent for detecting structurally abnormal proteins and the agent for reducing structurally abnormal proteins relate to the present invention can be used as an active ingredient of a pharmaceutical composition. They are useful as an active ingredient of a pharmaceutical composition used for treating or preventing a disease in which a structurally abnormal protein accumulates in a living body. In particular, many misfolded proteins in nerve tissue form aggregates that are believed to be the cause of neurodegenerative diseases, and reduction of the aggregates is expected to improve the pathological condition. Therefore, the pharmaceutical composition relate to the present invention is particularly preferred as an active ingredient of a pharmaceutical composition used for treating or preventing neurodegenerative diseases. In particular, since a loss-of-function mutation of hLONRF2 may be the cause of ALS, it is preferred as an active ingredient of a pharmaceutical composition used for treating or preventing ALS.

For example, a pharmaceutical composition can be prepared by appropriately mixing the agent for detecting structurally abnormal proteins or the agent for reducing structurally abnormal proteins relate to the present invention with a pharmaceutical acceptable carrier. The pharmaceutical composition can be produced by a method commonly used in the field of pharmaceutical production, using appropriate additives as necessary.

A pharmaceutical acceptable carrier is a diluent, excipient, binder, solvent and so on. that does not induce a harmful physiological reaction in the subject to administration and does not cause harmful interactions with other components such as medicinal ingredients. Specific examples of the carrier include water, physiological saline, various buffer solutions and so on. Also, examples of additives that can be used include adjuvants, diluents, excipients, binders, stabilizers, isotonicity agents, buffers, solubilizing agents, suspending agents, preservatives, cryoprotectants, cryoprotectants, lyophilization protectants, bacteriostatic agents and so on.

An animal to which the pharmaceutical composition relate to the present invention is administered is not particularly limited, and may be a human or a non-human animal. It is preferably a mammal. Examples of non-human mammals include cows, pigs, horses, sheep, goats, monkeys, dogs, cats, rabbits, mice, rats, hamsters, and guinea pigs. The route of administration of the pharmaceutical composition relate to the present invention to animals is not particularly limited, and may include oral administration, intravenous administration, enteral administration, intramuscular administration, subcutaneous administration, transdermal administration, nasal administration, and pulmonary administration.

As shown in Example 3 below, the abnormality of motor neurons observed in ALS is restored by normal LONRF2 functioning in motor neurons. Therefore, a pharmaceutical composition containing a functional nucleic acid for expressing the LONRF2 gene as an active ingredient is suitable as a pharmaceutical composition for treating or preventing ALS. ALS is caused by a low expression level of the LONRF2 gene due to gene mutation or the like, or by structural abnormality of the expressed LONRF2 protein. It can be expected that gene therapy, which introduces a normal LONRF2 gene into nerve cells, will improve the pathology of ALS. In particular, since ALS caused by an abnormality in the LONRF2 gene is delayed onset, there is a possibility that the onset itself can be suppressed by performing gene therapy to introduce the LONRF2 gene before the onset.

When used as an active ingredient of a pharmaceutical composition for treating or preventing ALS, the functional nucleic acid for expressing the LONRF2 gene may be incorporated into the genomic DNA, or may be present in a cell as an extracellular gene. In addition, it may be integrated into the host cell genomic DNA by replacing the entire length or a part of the LONRF2 gene that is originally present therein, or it may be newly integrated into the genomic DNA separately from the originally present LONRF2 gene.

Examples of functional nucleic acids for expressing the LONRF2 gene include (A1) a polypeptide consisting of the amino acid sequence represented by SEQ ID NO:1, or (B1) a polynucleotide encoding a polypeptide consisting of an amino acid sequence having 90% or more sequence identity with the amino acid sequence represented by SEQ ID NO:1 and having binding activity to a structurally abnormal protein and ubiquitin ligase activity, which is incorporated into an expression vector such as an adenovirus vector. The functional nucleic acid may be a polynucleotide containing not only the exons but also the introns of the LONRF2 gene incorporated into an expression vector such as an adenovirus vector. In addition, when a mutation in the LONRF2 gene of a patient to be treated is known, the normal LONRF2 gene can be expressed by replacing the mutated site in the LONRF2 gene with the same base as in the normal LONRF2 gene. In this case, for example, a polynucleotide encoding a polypeptide consisting of the amino acid sequence of a partial region of the normal LONRF2 gene corresponding to a partial region containing a mutated site in the LONRF2 gene, which is incorporated into an expression vector such as an adenovirus vector, can be used as a functional nucleic acid for expressing the LONRF2 gene.

### <ALS Model Animal>

As shown in the Examples below, knockout animals lacking the LONRF2 gene exhibit complex phenotypes such as movement disorders and cerebellar ataxia similar to those of ALS. This is caused by the accumulation of misfolded proteins generated in nerve cells without being degraded due to the deletion of LONRF2. For this reason, transgenic animals deleted the LONRF2 gene or having a mutation introduced into the LONRF2 gene that reduces its function are suitable as ALS model animals.

The introduction of a mutation that causes the deletion or functional impairment of the LONRF2 gene can be carried out by a conventional method using a known gene modification technique such as genome editing. An example of a mutation that causes the functional impairment of the hLONRF2 gene is the V599M mutation. The V599M mutant of hLONRF2 is a mutant that has lost the ability to bind to structurally abnormal proteins, and thus the degradation of structurally abnormal proteins is also suppressed.

A transformed animal into which a mutation that deletes or reduces the function of the LONRF2 gene has been introduced, and cells and tissues collected from the transformed animal are useful for screening of ALS therapeutic drugs and so on. For example, stem cells such as iPS cells and mesenchymal stem cells prepared from somatic cells such as fibroblasts of the transformed animal, and nerve cells induced to differentiate from these stem cells are also useful for screening of ALS therapeutic drugs and so on.

Transformed cells in which the LONRF2 gene has been deleted or a mutation that reduces its function has been introduced in primary subculture cells of neurons that have the LONRF2 gene and cultured cells derived from those neurons can also be used as ALS models. For example, transformed cells obtained by deleting the LONRF2 gene or introducing a mutation that reduces its function from human-derived cultured cells are useful for screening therapeutic agents for ALS and so on.

### <A Method for Evaluating the Risk of Developing a Disease>

The V599M mutant, which is a loss-of-function mutant of LONRF2, is generated by a single base substitution mutation of rs143848902. When the rs143848902 genotype is GTG type, the 599^{th} amino acid of hLONRF2 is wild-type valine, but when the rs143848902 genotype is ATG type, the 599^{th} amino acid of hLONRF2 is methionine. Therefore, the risk of developing a disease caused by loss of function of LONRF2 can be evaluated based on the genotype of rs143848902.

Specifically, the method for evaluating the risk of developing a disease relate to the present invention includes a typing step of typing the genotype of rs143848902 of a human subject, and an evaluation step of evaluating the risk of developing a disease in which abnormal proteins accumulate in the body of the subject based on the typing result obtained by the typing step. If the genotype of rs143848902 of the subject is the ATG type, the subject is evaluated as having a high risk of developing the disease. The genotype can be typed by a conventional method in gene analysis. This evaluation method is particularly effective for evaluating the risk of developing ALS.

### EXAMPLES

Next, the present invention will be described in more detail with reference to examples, but the present invention is not limited to these examples.

### <Cell Culture>

Early passage human fibroblast HCA2 cells, A549 cells (obtained from ATCC, CCL-185), MCF7 cells (obtained from ATCC, HTB-22), AsPC1 cells (obtained from ATCC, CRL-1682), and 293T cells (obtained from ATCC, ACS-4500) were cultured in Dulbecco's modified Eagle's medium (DMEM) supplemented with 10% fetal bovine serum (FBS) at 37° C. under 5% CO₂.

For the cycloheximide chase assay, cells were treated with 100 mg/mL cycloheximide (Sigma-Aldrich).

Cortical neurons were prepared as follows: First, cerebral cortices of E15.5 C57BL6 mouse embryos were digested with 2.4 U/mL papain (Worthington) and 0.01% DNase I (Roche Life Science) at 37° C. for 60 min. Next, cells in the digest were plated on poly-L-lysine-coated 10 cm dishes at a density of 5.5×10⁴ cells/cm² and maintained in neurobasal medium (Invitrogen) containing 2% B27, 2 mM glutamine, 50 units/mL penicillin, 25 mg/mL streptomycin, 25 mM glutamate, 25 mM 2-mercaptoethanol, and 1% FCS in 5% CO₂, H₂O-saturated atmosphere at 37° C. for 1 day and 14 days.

### <Senescence Induction>

Fibroblast senescence induction was prepared as follows: First, HCA2 cells were treated with 9 mM R03306 (Roche) for 24 hours, followed by 9 mM R03306 and 5 mM nutlin3a (Sigma-Aldrich) for 8 hours, and then 5 mM nutlin3a for 1.5 days to synchronize them in the G2 phase. Proliferating cells were then eliminated by treatment with 100 nM BI-2536 for 9 days, and then cultured in normal medium for another 9 days.

### <Plasmid Construction>

Lentivirus-based shRNA constructs targeting LONRF2 and Tet-on inducible lentivirus constructs were generated by using two types of shRNA target sequences (shLONRF2-1 and shLONRF2-2). As a control, a similar construct was generated by using an shRNA target sequence targeting luciferase (shControl).

TABLE 3

| shRNA | Target sequence | Seq. No. |
|---|---|---|
| shLONRF2-1 | GGAGCUGGCUCCUGAUGAUAA | 7 |
| shLONRF2-2 | GCUGCGGGCGGAGUUAUAUUU | 8 |
| shControl | CGUACGGAAUACUUCGA | 9 |

To generate lentivirus-based shRNA constructs, a 19-21 base shRNA-coding fragment with a 5′-ACGTGTGCTGTCCGT-3′ loop was introduced into pENTR4-H1 (provided by RIKEN) digested with Agel/EcoRI. To insert the HltetOx1-shRNA into the lentivirus vector, we mixed the resulting pENTR4-H1-shRNA vector and CS-RfA-ETBsd vector (provided by RIKEN) with Gateway LR clonase (Invitrogen). To construct Tet-on-inducible lentivirus constructs, the PCR-generated EcoRI/NotI fragments containing cDNA for human FLAG-LONRF2-WT were inserted into a pENTR-1A vector (Invitrogen) containing the FLAG-tag digested with EcoRI/NotI. The resultant plasmid was mixed with CS-IV-TRE-RfA-UbC-Puro vector42, and reacted with Gateway LR clonase to generate the lentivirus plasmid.

To construct lentivirus plasmid constitutively expressing the transgenes (LONRF2, FLAG-LONRF2, Fluc-HA-WT, Fluc-HA-DM, NLS-AgDD-HA, FLAG-IDP, or FLAG-hnRNP M1), the PCR-generated EcoRI/BanHI fragments containing cDNA for each gene were inserted into a CSII-CMV-IRES2-Bsd vector (provided by RIKEN) digested with EcoRI/BamHI. pcDNA3-(HA-Ub)×6 containing six tandem repeats of HA-tagged ubiquitin. As LONRF2, the wild type (LONRF2-WT), a mutant LONRF2-RINGm (C4A) in which four amino acid mutations (C4A: C143A, C146A, C499A, C452A) (Non Patent Literature 19) have been introduced into the RING finger domain, and a mutant LONRF2-LonSBm (P5A) in which five amino acid mutations (P5A: P539A, P548A, P553A, P603A, P707A) (Non Patent Literature 20) have been introduced into the LonSB domain were similarly prepared. pcDNA3-(HA-Ub)×6 containing six tandem repeats of HA-tagged ubiquitin was used as previously described (Non Patent Literature 18). To construcl a plasmid expressing the indicated lonrf2 mutants, CSII-CMV-IRES2-Bsd-lonrf2 or FLAG-lonrf2 was modified with a KOD-plus-mutagenesis kit (TOYOBO). The Fluc-Wt-HA-GFP11-N1 (Addgcnc, 9195446), FlucDM-HA-GFP11-N1 (Addgcnc, 9195646), and NLS-AgDD (Addgcnc, 8062534) were used.

### <Virus Generation and Infection>

Generation of lentiviruses and their infection of cells were performed as follows. First, Lentiviruses expressing the respective shRNAs or genes were generated by co-transfection of 293T cells with pCMV-VSV-G-RSV-RevB, pCAG-HIVgp, and the respective CS-RfA-ETBsd, CS-IV-TRE-RfA-UbC-Puro, or CSII-CMV-IRES2-Bsd using the calcium phosphate co-precipitation method. Cells infected with the indicated viruses were treated with 10 mg/mL of blasticidin (Thermo Fisher Scientific) for 2-3 days. Doxycycline (Hereinafter referred to as “Dox”; Sigma Aldrich) was added to the medium at a concentration of 1 mg/mL for inducible expression of the respective shRNAs or genes.

### <RNA-Seq Library Preparation and RNA Sequencing>

Total RNA was extracted and purified from cells using an RNeasy mini kit (Qiagen). The integrity of the RNA was assessed using an Agilent 2100 Bioanalyzer (Agilent Technologies), and total RNAs with RIN (RNA Integrity Number)>7.5 were used for subsequent steps. Ribosomal RNA (rRNA) was removed from 1 μg of total RNA using RiboMinus Eukaryote System v2 (Thermo Fisher Scientific). RNA-seq libraries were prepared using the rRNA-depleted RNAs with an Ion Total RNA-Seq Kit v2 (Thermo Fisher Scientific) according to the manufacturer's instructions. The libraries were sequenced on an Ion Proton instrument using an Ion PI Sequencing 200 Kit v3 and Ion PI Chip Kit v2 (Thermo Fisher Scientific). The sequencing data were analyzed using Torrent Suite v5.0.2 with a plug-in “RNASeqAnalysis v5.0.2.1” program. The sequencing reads were aligned to hg19 using “STAR (v2.3.0e)” and “Bowite2 (v2.0.0-beta7)”. Read counts were obtained using “HTSeq (v0.5.3p9)”. The “package edgeR” was used for normalization and analysis of differentially expressed genes. Differentially expressed genes were selected based on FDR (q<0.05) and the log2 fold change (>4) on the volcano plot.

<Quantitative-PCR (qPCR)>

qPCR was performed as follows. Total RNA from the cell culture was extracted using an “RNeasy Mini Kit” (QIAGEN) according to the manufacturer's instructions.

Total RNA from cultured cortical neurons were extracted as follows. First, cells were lysed with 1 mL of TRIzol Reagent (Invitrogen) by pipetting and collected into a 2 mL tube. After shearing the nuclear DNA by moving the 1 mL syringe with a 21G needle up and down, the homogenates were stored at −80° C. After thawing, the RNAs were extracted and purified using an RNAeasy mini kit (Qiagen) according to the manufacturer's instructions, and then stored at −80° C.

For qPCR analysis, cDNAs were synthesized using a ReverTra Ace qPCR kit (TOYOBO). An MTC Mouse Panel (Takara) was used for cDNA from mouse tissues.

Real-time PCR amplifications were performed in 96-well optical reaction plates with “Power SYBR Green PCR Master Mix” (Applied Biosystems). The relative expression values of each gene were determined by normalization to GAPDH expression for each sample. For real-time PCR, primers consisting of the following base sequences were used.

TABLE 4

| Primer | Sequence | Seq. No. |
|---|---|---|
| Human lonrf2-forward | GGCGTTAGAAAGCATCCTTCC | 11 |
| primer | | |
| Human lonrf2-reverse | GCAGAGGGCACACTCAAAGT | 12 |
| primer | | |
| Human p16-forward | CCCAACGCACCGAATAGTTA | 13 |
| primer | | |
| Human p16-reverse | ACCAGCGTGTCCAGGAAG | 14 |
| primer | | |
| Human GAPDH-forward | GGAGCGAGATCCCTCCAAAAT | 15 |
| primer | | |
| Human GAPDH-reverse | GGCTGTTGTCATACTTCTCATGG | 16 |
| primer | | |
| Mouse lonrf2-forward | ACCAAGGGGCACCATATAAAAG | 17 |
| primer | | |
| Mouse lonrf2-reverse | CTTCGCCGAGTTGCACTCA | 18 |
| primer | | |
| Mouse p16-forward | CGCAGGTTCTTGGTCACTGT | 19 |
| primer | | |
| Mouse p16-reverse | TGTTCACGAAAGCCAGAGCG | 20 |
| primer | | |
| Mouse GAPDH-forward | CATCACTGCCACCCAGAAGACTG | 21 |
| primer | | |
| Mouse GAPDH-reverse | ATGCCAGTGAGCTTCCCGTTCAG | 22 |
| primer | | |

### <Immunofluorescence Staining>

Cells on glass bottom dishes were fixed in 4% paraformaldehyde for 10 minutes at room temperature, and permeabilized with 0.2% TritonX-100 in PBS for 5 minutes at room temperature, and then were incubated with blocking buffer (5% BSA in PBS) for 30 minutes. The blocked cells were then incubated with a primary antibody at room temperature for 2 hours, and then with a secondary antibody at room temperature for 1 hour. Images of cells after fluorescent immunostaining were taken using a fluorescent microscope (Keyence, BZ-9000). Representative images were similarly processed with BZ-II analysis software (Keyence).

For fluorescent immunostaining of G3BP1, a marker for stress granule inclusions, anti-G3BP1 mouse antibody (Abeam, 2F3 clone, 1/100 dilution) was used as the primary antibody, and anti-mouse lgG antibody conjugated with Alexa Fluor 488 (Life Technologies) or Alexa Fluor 555 (Life Technologies) ( 1/200 dilution) was used as the secondary antibody. For fluorescent immunostaining of FLAG-tagged proteins, anti-FLAG mouse antibody (Sigma-Aldrich, M2, 1/100 dilution) was used as the primary antibody, and the same antibody as above was used as the secondary antibody. For nuclear fluorescent staining, cells were detected by counterstaining with the nuclear stain Hoechst 33342 (1 mg/mL) (Enzo).

### <Stress Granule Formation Assay>

For stress granule formation assays, cells on glass bottom dishes were treated with sodium arsenite (NaAsO₂) (1 mM, for 30 minutes), H₂O2 (1 mM, for 60 minutes), or heat shock (43° C., for 60 minutes), and were recovered at specific times. The recovery treatment was carried out by incubating the cells in a buffer or culture medium that did not contain sodium arsenite or hydrogen peroxide, and by incubating the cells at a normal culture temperature (37° C.) for the heat shock treatment.

### <ProteoStat Staining>

Cells on glass bottom dishes (Greiner Bio-One, “CELLview (registered trademark) Sterile glass bottom dish”) were fixed in 4% paraformaldehyde for 10 minutes at room temperature, washed with PBS, and then permeabilized with 0.2% TritonX-100 in PBS for 5 minutes at room temperature. The permeabilized cells were incubated in a 1/2000 dilution of “ProteoStat (registered trademark) Aggregome Detection Reagent” (Enzo) for nuclear staining. After extensive washing of the stained cells with PBS, fluorescent staining images were taken, and the fluorescence intensity of each cell was quantified using an image analyzer “IN Cell Analyzer 2500HS” (GE Healthcare). Statistical analysis was performed by averaging the intensities of 200 cells per sample.

### <Luciferase Assay>

HeLa cells were transfected with the indicated plasmid and pCMV-NanoLuc (Promega) for normalization using Lipofectamine3000 (Invitrogen). After 48 hours, cells were treated with 100 mg/mL cycloheximide (CHX)(Sigma-Aldrich) for 6 hours. The luciferase activity of the obtained cells was measured with the “Dual-Luciferase Reporter Assay System” (Promega, E1910) according to the instructions provided by the manufacturer.

### <Immunoprecipitation and Immunoblotting Analyses>

Immunoprecipitation and immunoblotting were performed as follows. Cells were lysed in tris-buffered saline containing NP-40 (TBSN) buffer {20 mM Tris-Cl (pH8.0), 150 mM NaCl, 0.5% NP-40, 5 mM EGTA, 1.5 mM EDTA, and 0.5 mM Na₃VO₄}. The resulting lysates were clarified by centrifugation at 15,000×g for 20 minutes at 4° C. before immunoprecipitation with the specified antibody. For whole lysates, cells or tissues were directly lysed with Laemmli-buffer (2% SDS, 10% glycerol, 5% 2-mercaptoethanol, 0.002% bromophenol blue, and 62.5 mM Tris HCl at pH 6.8). The whole lysates (20-50 mg) were separated by SDS-PAGE, transferred to a PVDF (Immobilon-P; Millipore) membrane, and then subjected to immunoblotting with the appropriate antibodies using the ECL detection system. The primary antibodies used were mouse anti-β-actin antibody (Santa Cruz Biotechnology, AC-15), rabbit anti-FLAG antibody (Cell Signaling Technology, D6W5B), rabbit anti-HA antibody (Cell Signaling Technology, c29F4), rabbit anti-hRNP M1-4 antibody (Abeam, EPR13509B), and rabbit anti-TDP43 (Cell Signaling Technology, G400).

### <Ubiquitination Assay>

First, plasmids were transiently transfected into cells using Lipofectamine3000 (Invitrogen). After 48 hours, cells were lysed in lysis buffer (50 mM Tris-HCl, pH 7.5, 300 mM NaCl, 0.5% Triton X-100) containing protease inhibitors (Roche) and a deubiquitinase inhibitor (Sigma Aldrich). An equal volume of 2× denaturing IP buffer (100 mM Tris-HCl, pH 7.5, 2% SDS, 10 mM DTT) was added to the cell lysates which were then incubated at 100° C. for 10 minutes and centrifuged at 15,000×g at room temperature for 10 minutes. The supernatants were diluted with five volumes of lysis buffer and were immunoprecipitated with the indicated antibodies at 4° C. followed by immunoblotting.

### <FLAG-LONRF2 Pull-Down Assay>

For preparing FLAG-LONRF2 beads, FLAG-LONRF2 was expressed in SF9 insect cells and affinity-purified with FLAG M2 agarose gel (Sigma-Aldrich) in the same manner as in the above<Immunoprecipitation and immunoblotting analysis>. For the pull-down assay, cells were harvested with TBSN buffer. Lysates (500 mg) were incubated with 30 mL of FLAG-LONRF2 beads for 1 hour at 4° C. Proteins bound to the beads were washed with TBSN buffer, separated by SDS-PAGE, and analyzed by immunoblotting with appropriate antibodies.

### <LONRF2 Antibody Production>

Antiserum against human LONRF2 were raised in rabbits by immunization with a GST-tagged recombinant full-length human LONRF2 (Hokudo). Furthermore, antisera were further affinity-purified using FLAG-LONRF2 immobilized with NHS-activated “Sepharose4 Fast Flow” (GE Healthcare) and used for immunoblotting analyses.

### <Statistics>

In the following experiments, unless otherwise specified, results were expressed as mean±SD (standard deviation) or percentage. Comparisons of results of statistical analysis were performed by Student's t-test, a log-rank(Mantel-Cox) test, and one-way/two-way ANOVA with Tukey or Dunnett's multiple comparison post-hoc tests for independent biological replicates acquired after testing for homogeneity of variances (Prism8 or 9). A probability value of <0.05 was regarded as a difference with statistical significance (*P<0.05 vs. control, **P<0.01 vs. control, ***P<0.001 vs. control, ****P<0.0001 vs. control). For all representative findings, the results of triplicate experiments were similar.

### Example 1

We identified a gene that functions as a mammalian nuclear PQC ligase and investigated its effect on structurally abnormal proteins.

### (1) Identification of Genes Whose Expression is Upregulated in Senescent Cells

In post-mitotic cells, the contents of nuclear and cytosolic compartments never have the chance to intermingle. The inventors of the present invention speculated that the nucleus may be equipped with a unique nuclear PQC system to mitigate the damage caused by misfolded proteins, and that differentiation of mitotic into post-mitotic cells might induce genes that are involved in nuclear PQC. On the other hand, induction of senescence causes a switch from the mitotic to postmitotic phase in almost all cells, and increases the amount of protein aggregates (Non Patent Literature 17). Therefore, the present inventors considered that the gene whose expression is induced in senescent cells is highly likely to be a gene involved in nuclear PQC, and attempted to identify said gene.

Senescence was induced in human fibroblast HCA2 cells by treatment with Nutlin3a, and an RNA-seq library was prepared from total RNA extracted from pre-senescence induced HCA2 cells and senescence induced HCA2 cells. RNA-seq data have been deposited in the Gene Expression Omnibus (GEO) under accession number GSE179465. The RNA in the library was sequenced to examine genes whose expression levels changed between cells before and after senescence induction. As a result, it was found that the expression of LONRF2, a RING finger type E3 ligase with a LonSB domain, was significantly induced in HCA2 cells in which senescence was induced by nutlin3a. The expression levels of LONRF1 and LONRF3, the remaining two of the three LONRF isozymes in mammals, did not increase with senescence induction.

LONRF2 expression was also induced in senescent HCA2 cells induced by doxorubicin treatment. These results suggest that LONRF2 may be a mammalian PQC ubiquitin ligase that plays a role in suppressing misfolding in postmitotic cells.

When LONRF2 was overexpressed in cultured cells, wild-type LONRF2 was found to be mainly localized in the nucleus and also present in the cytoplasm. The intracellular localization of LONRF2-RINGm(C4A) and LONRF2-LonSBm(P5A) was not different from that of LONRF2-WT, indicating that mutations in the RING finger domain or LonSB domain do not affect the localization of LONRF2.

Next, the tissue distribution of LONRF2 in normal conditions was examined by qPCR analysis, and LONRF2 was mainly expressed in the brain. In addition, single-cell analysis using a dataset of aged mouse brains (Ximerakis et al., Nature Neuroscience, 2019, vol. 22, p. 1696-1708) revealed that LONRF2 is mainly expressed in mature neurons.

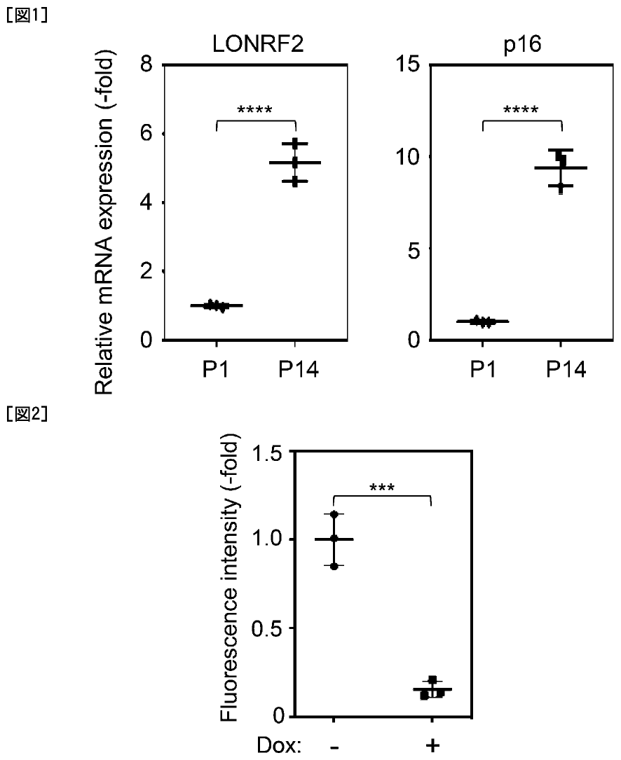

In addition, qPCR analysis was performed using RNA from primary cerebral cortical neuron cells cultured for 1 or 14 days. The results of relative LONRF2 expression are shown in FIG. 1. Data are shown as the mean±s.d. of three independent experiments. Statistical analysis was performed by paired two-tailed Student's t-test. In FIG. 1, “P1” is the result of cells cultured for 1 day, and “P14” is the result of cells cultured for 14 days. As shown in FIG. 1, it was found that the expression of LONRF2, like the aging marker protein p16, was significantly increased when primary neuronal cells were cultured for a long period of time.

### (2) Effect of LONRF2 on Protein Aggregation

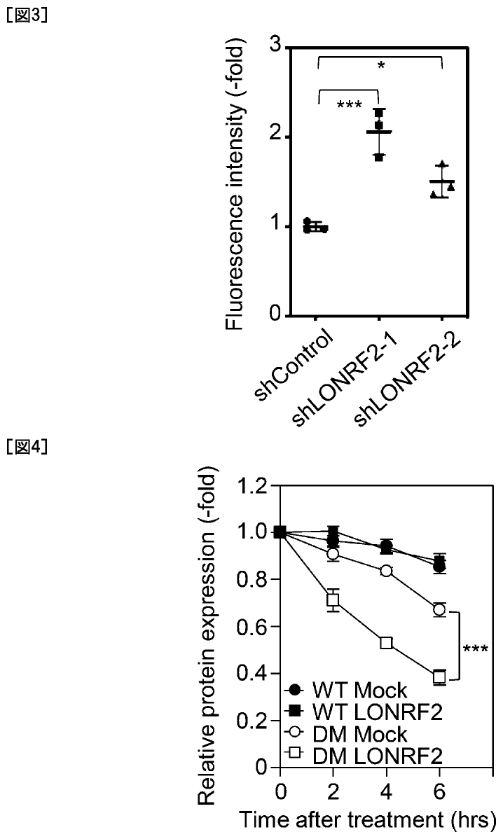

Next, the effect of LONRF2 on protein aggregation was examined. First, d-Sen cells expressing FLAG-LONRF2 by Dox induction were stained with a proteostat in the presence or absence of Dox (1 mg/mL). The measurement results of the fluorescence intensity of each cell are shown in FIG. 2. Data are shown as the mean±s.d. of three independent experiments. Statistical analysis was performed by two-tailed Student's t-test without a control table. As a result, the fluorescence intensity was significantly lower in cells overexpressing FLAG-LONRF2 in the presence of Dox (in the Figure, “+”) than in cells without Dox (in the Figure, “−”), and protein aggregation was significantly suppressed by overexpression of FLAG-LONRF2. On the other hand, d-Sen cells expressing shRNA (shLONRF2-1, shLONRF2-2, or shControl) by Dox induction were cultured in the presence of Dox (1 mg/mL), and the fluorescence intensity was measured. The results are shown in FIG. 3. Data are shown as the mean±s.d. of three independent experiments. Statistical analysis was performed by one-way ANOVA and Dunnett's post-hoc test for multiple comparisons. As a result, in cells expressing shLONRF2-1 or shLONRF2-2, the fluorescence intensity by proteostat staining was significantly enhanced compared to cells expressing shControl, and protein aggregation was significantly promoted by suppression of LONRF2 expression.

### (3) Effect of LONRF2 on Fluc-DM Protein

To confirm whether LONRF2 is a mammalian PQC ubiquitin ligase, experiments were carried out using the R188Q/R261Q double mutant (Fluc-DM) of firefly luciferase (Flue), a model for controllable misfolded proteins (Non Patent Literature 21).

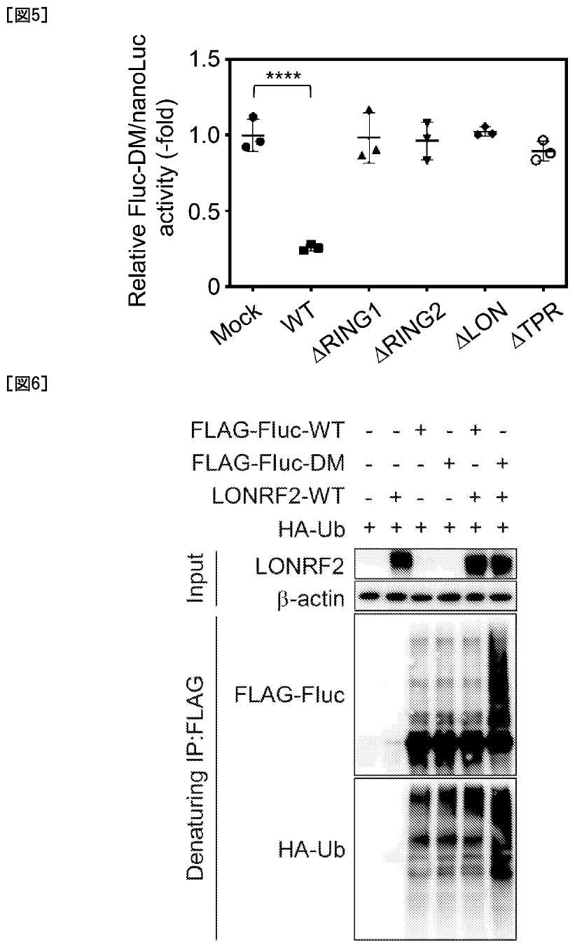

First, FLAG-LONRF2-WT or mock was co-expressed with HA-tagged wild-type luciferase (Fluc-HA-WT) or HA-tagged Fluc-DM (Fluc-HA-DM) in HeLa cells. The cells were cultured in the presence of 100 mg/mL CHX for 0 to 6 hours and then lysed. The obtained cell lysates were subjected to immunoblotting using anti-HA antibodies, and the HA staining intensities were quantified using the image analysis software “ImageJ”. The results are shown in FIG. 4. Data are shown as the mean value±s.d. of three independent experiments. Statistical analysis was performed by two-tailed Student's t-test without control table. As a result, the intracellular abundance of Fluc-WT was not affected by co-expression of LONRF2-WT, but the intracellular abundance of Fluc-DM was reduced (FIG. 4). These results demonstrated that LONRF2-WT did not affect the intracellular stability of wild-type luciferase, which was a normal protein, but reduces the protein amount of Fluc-DM, which was a structurally abnormal protein due to misfolding.

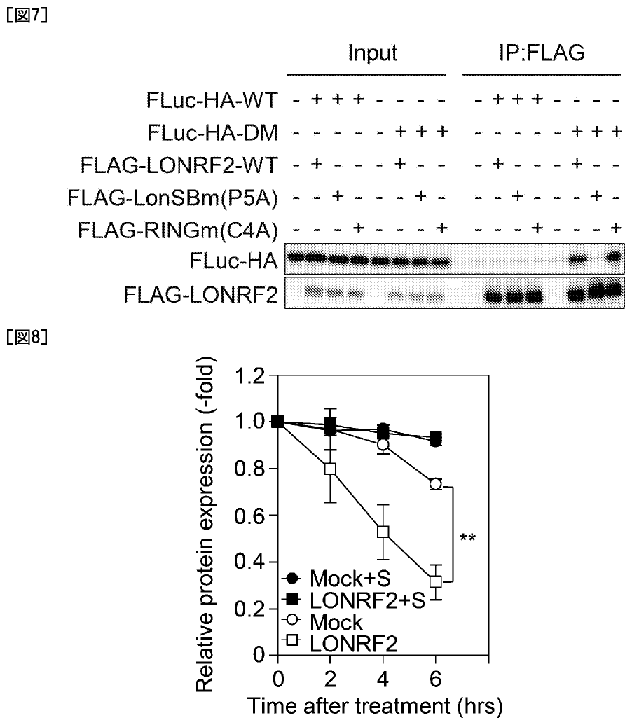

In addition, FLAG-LONRF2-WT, FLAG-LONRF2-RINGm (C4A), FLAG-LONRF2-LonSBm (P5A), or mock was co-expressed with Fluc-HA-WT or Fluc-HA-DM in HeLa cells, and the cells were cultured in the presence of 100 mg/mL CHX for 6 hours, and then dissolved to prepare cell lysates. A luciferase assay was also performed on the prepared cell lysates, and luciferase activity was significantly reduced only in the lysates of cells co-expressing LONRF2-WT and Fluc-DM. Furthermore, luciferase assay was performed similarly on lysates of cells in which FLAG-tagged LONRF2 lacking the TPR domain (FLAG-LONRF2-ΔTPR), FLAG-tagged LONRF2 lacking the RING finger domain 1 (FLAG-LONRF2-ΔRING1), FLAG-tagged LONRF2 lacking the RING finger domain 2 (FLAG-LONRF2-ΔRING2), or FLAG-tagged LONRF2 lacking the LonSB domain (FLAG-LONRF2-ΔLonSB) were co-expressed with Fluc-DM instead of FLAG-LONRF2-WT. The results are shown in FIG. 5. Data are shown as the mean±s.d. of three independent experiments. Statistical analysis was performed by one-way ANOVA followed by Dunnett's post hoc test for multiple comparisons. As a result, a reduction in luciferase activity similar to that observed in LONRF2-WT was not observed in any of the LONRF2 deletion mutants. These results demonstrated that the reduction in the amount of Fluc-DM protein by LONRF2 requires not only the TPR domain but also both the two RING finger domains and the LonSB domain.

Next, to examine whether LONRF2 specifically ubiquitinates misfolded Flue, an in vivo ubiquitination assay was performed. Specifically, HeLa cells were co-expressed LONRF2-WT and HA-Ub with FLAG-Fluc-WT or FLAG-Fluc-DM, and then cultured for 48 hours. The HeLa cells were lysed in a lysis buffer containing a protease inhibitor and a deubiquitinase inhibitor. The obtained cell lysate was incubated with an equal amount of 2× denaturing IP buffer, and then immunoprecipitated with anti-FLAG M2 affinity gel and immunoblotted with an anti-FLAG antibody. The results are shown in FIG. 6. LONRF2-WT specifically ubiquitinates Fluc-DM, but not Fluc-WT. On the other hand, when LONRF2-RINGm(C4A) or LONRF2-LonSBm(P5A) was expressed instead of LONRF2-WT and a similar in vivo ubiquitination assay was performed, neither LONRF2-RINGm(C4A) nor LONRF2-LonSBm(P5A) ubiquitinated Fluc-DM.

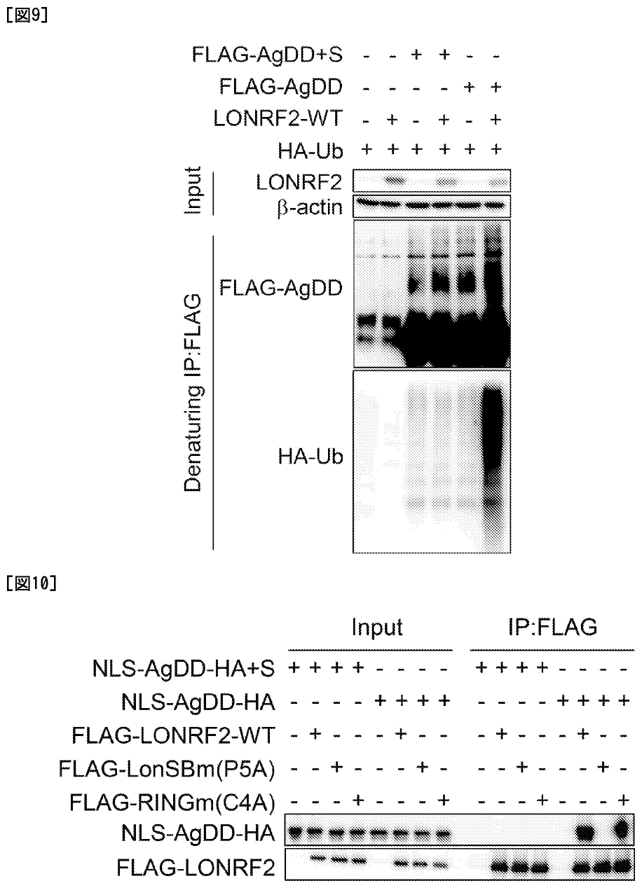

HeLa cells were co-expressed Fluc-HA-WT or Fluc-HA-DM with FLAG-LONRF2-WT, FLAG-LONRF2-RINGm (C4A) or FLAG-LONRF2-LonSBm (P5A), and then cultured for 48 hours. The cell lysates of the HeLa cells were immunoprecipitated with anti-FLAG M2 affinity gel, and then immunoblotted with anti-FLAG antibodies. The results are shown in FIG. 7. As a result, LONRF2-WT, which ubiquitinated Fluc-DM, bound to Fluc-DM, but did not bind to Fluc-WT, which was not ubiquitinated (FIG. 7). Furthermore, LONRF2-RINGm(C4A) bound to Fluc-DM, but LONRF2-LonSBm(P5A) did not bind to Fluc-DM (FIG. 7). These results confirmed that LONRF2 binds to the structurally abnormal protein Fluc-DM via the LonSB domain and ubiquitinates it, and that the LonSB domain of LONRF2 does not bind to the normal protein Fluc-WT, nor does it ubiquitinate Fluc-WT.

### (4) Effect of LONRF2 on Aggregation-Prone Protein (AgDD-S)

To confirm whether LONRF2 is a PQC ubiquitin ligase, immunoblotting was performed using the aggregation destabilization domain protein (AgDD) (Non Patent Literature 22). AgDD-S is a chemically controllable system, and withdrawal of the small molecule drug “Shield-1” (CAS No: 914805-33-7) rapidly induces the formation of AgDD aggregates in cells.

First, FLAG-LONRF2-WT or mock was co-expressed with NLS-AgDD-HA in HeLa cells. The cells were cultured for 0 to 6 hours in the presence of 100 mg/mL CHX or in the presence of 100 mg/mL CHX and Shield-1, and then lysed. The obtained cell lysates were subjected to immunoblotting using anti-HA antibodies, and the HA intensity was quantified using the image analysis software ImageJ. The results are shown in FIG. 8. Data are shown as the mean±s.d. of three independent experiments. Statistical analysis was performed by a two-tailed Student's t-test without a control table. As a result, when cultured in the absence of Shield-1, the intracellular abundance of NLS-AgDD-HA was reduced in cells co-expressing LONRF2-WT (FIG. 8). These results indicate that LONRF2-WT does not affect the intracellular stability of pre-aggregated AgDD, but reduces the protein amount of AgDD aggregates, which are structurally abnormal proteins.

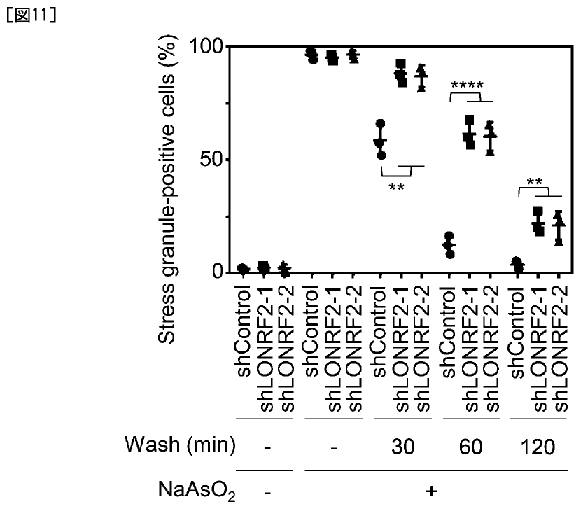

Next, to examine whether LONRF2 specifically ubiquitinates AgDD aggregates, an in vivo ubiquitination assay was performed. Specifically, LONRF2-WT, FLAG-AgDD, and HA-Ub were co-expressed in HeLa cells, and the HeLa cells were cultured for 48 hours in the presence or absence of Shield-1 and lysed with a lysis buffer containing a protease inhibitor and a deubiquitinase inhibitor. The obtained cell lysate was incubated with an equal amount of 2× denaturing IP buffer, and then immunoprecipitated with anti-FLAG M2 affinity gel and immunoblotted with anti-FLAG antibody. The results are shown in FIG. 9. LONRF2-WT specifically ubiquitinated AgDD aggregates formed in cells cultured in the absence of Shield-1, but did not ubiquitinate non-aggregated AgDD in cells cultured in the presence of Shield-1

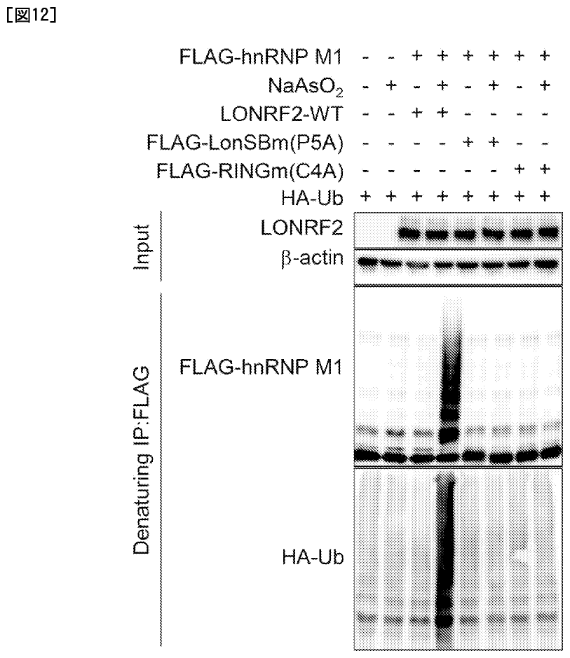

NLS-AgDD-HA was co-expressed with FLAG-LONRF2-WT, FLAG-LONRF2-RINGm (C4A) or FLAG-LONRF2-LonSBm (P5A) in HeLa cells, and the HeLa cells were cultured for 48 hours in the presence or absence of Shield-1. The cell lysates were immunoprecipitated with anti-FLAG M2 affinity gel, and then immunoblotted using anti-FLAG antibodies. The results are shown in FIG. 10. As a result, in the presence of Shield-1, NLS-AgDD-HA and LONRF-WT did not bind, whereas LONRF-WT bound to AgDD aggregates formed in the absence of Shield-1 (FIG. 10). Furthermore, LONRF2-RINGm(C4A) bound to AgDD aggregates, but LONRF2-LonSBm(P5A) did not bind to AgDD aggregates (FIG. 10). These results confirmed that LONRF2 binds to AgDD aggregates, which are structurally abnormal proteins, via the LonSB domain and ubiquitinates them, and that the LonSB domain of LONRF2 does not bind to or ubiquitinate non-aggregated AgDD.

### (5) Effect of LONRF2 on Cytoplasmic Stress Granules (SGs)

The results of (1) to (4) above indicate that LONRF2 is a PQC ubiquitin ligase that does not bind to normal proteins but binds to structurally abnormal proteins and ubiquitinates them. In recent years, a PQC network that controls the dynamics of cytoplasmic stress granules has been proposed. Stress granules accumulate efficiently upon treatment with sodium arsenite and are rapidly degraded upon removal of sodium arsenite. These stress granules can be detected using an anti-G3BP1 antibody. Therefore, we investigated how LONRF2 acts on the dynamics of these stress granules.

First, d-Sen cells were prepared by introducing Dox-inducible shRNA (shLONRF2-1, shLONRF2-2, or shControl) into A549 cells, which have a relatively high expression level of LONRF2. These d-Sen cells were cultured in the presence of Dox (1 mg/mL), and the cell lysates were subjected to Western blotting using an anti-LONRF2 antibody. It was confirmed that the expression level of LONRF2 was significantly reduced in cells introduced with Dox-inducible shLONRF2-1 and Dox-inducible shLONRF2-2 compared to cells introduced with Dox-inducible shControl.

Next, these d-Sen cells were cultured in the presence of Dox (1 mg/mL) for 48 hours, then incubated in the presence of 1 mM sodium arsenite for 30 minutes, and then washed (recovery) in PBS for 30, 60, or 120 minutes. After washing, the cells were subjected to fluorescent immunocytochemical staining using anti-G3BP1 antibodies to observe G3BP1-positive foci (proteins aggregates stained with anti-G3BP1 antibodies). As controls, cells that were not treated with sodium arsenite or washed, and cells that were treated with sodium arsenite and then not washed were similarly subjected to fluorescent immunocytochemical staining using anti-G3BP1 antibodies. Cells containing 5 or more G3BP1-positive foci were determined to be stress granule-positive cells.

The percentage (%) of stress granule-positive cells in the total cells (n=200) of d-Sen cells subjected to each treatment is shown in FIG. 11. In cells transfected with Dox-inducible shControl, G3BP1-positive foci were rapidly degraded by washing treatment, and stress granule-positive cells were almost completely eliminated by 120 minutes of washing. In contrast, in cells transfected with Dox-inducible shLONRF2-1 or Dox-inducible shLONRF2-2, the rate of decrease in stress granule-positive cells was very slow, and the decomposition process of stress granules was dramatically impaired by suppression of LONRF2 expression. When LONRF2-WT was further co-expressed in cells transfected with Dox-inducible shLONRF2-1, the ability to degrade stress granules was restored, whereas when LONRF2-RINGm (C4A) or LONRF2-LonSBm (P5A) was co-expressed, the ability to degrade stress granules was not restored.

Even when hydrogen peroxide treatment or heat shock treatment was performed instead of sodium arsenite treatment, the decomposition of stress granules was similarly inhibited by suppression of LONRF2 expression. Furthermore, similarly to A549 cells, in MCF7 cells and AsPC-1 cells, which are highly LONRF2 expressing cells, the decomposition of stress granules formed by sodium arsenite treatment, hydrogen peroxide treatment, and heat shock treatment was similarly inhibited by suppression of LONRF2 expression.

### Example 2

Since LONRF2 is mainly expressed in the nervous system, the effect of LONRF2 on misfolded proteins in neurons was investigated. As misfolded proteins in neurons, hnRNP M1 or TDP43, which are heterogeneous nuclear ribonucleoproteins (hnRNPs), were used after treatment with sodium arsenite.

(1) Effect of LONRF2 on Ubiquitination of hnRNP M1

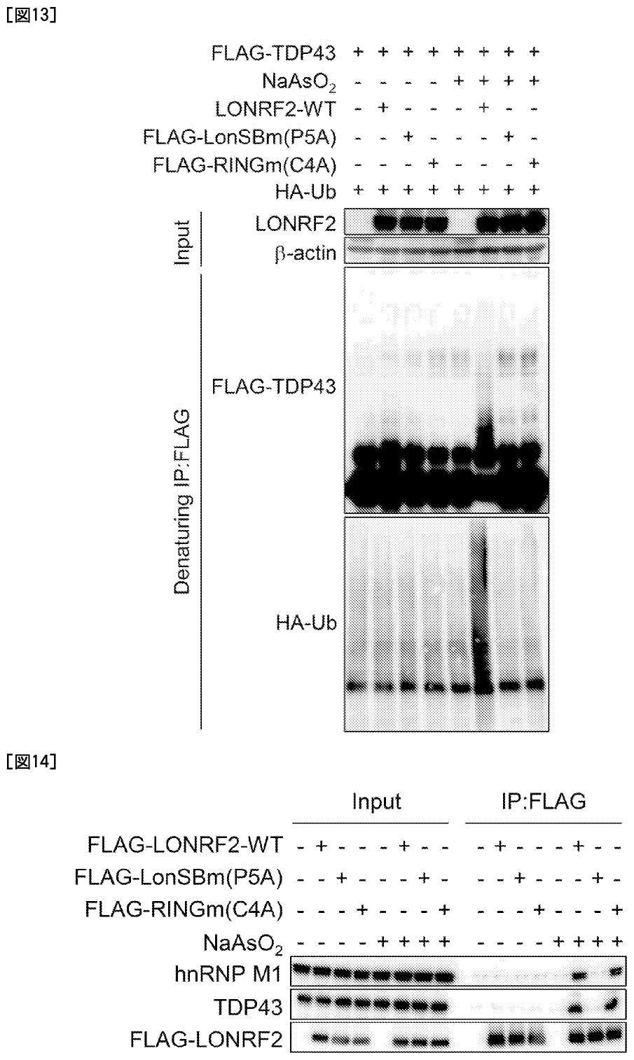

To examine whether LONRF2 ubiquitinates the abnormally structured protein of hnRNP M1, an in vivo ubiquitination assay was performed. Specifically, A549 cells co-expressing FLAG-hnRNP M1, and HA-Ub with LONRF2-WT, FLAG-LONRF2-RINGm (C4A), or FLAG-LONRF2-LonSBm (P5A) were incubated in the presence of 1 mM sodium arsenite for 30 minutes, and then the cells were solubilized by adding denaturing IP buffer. The obtained cell lysates were immunoprecipitated with anti-FLAG M2 affinity gel and immunoblotted with anti-FLAG antibodies. The results are shown in FIG. 12. In A549 cells overexpressing hnRNP M1, misfolded hnRNP M1 proteins induced by sodium arsenite treatment were specifically ubiquitinated only when LONRF2-WT was co-expressed (FIG. 12). Ubiquitination of misfolded proteins was not observed when LONRF2-RINGm(C4A) or LONRF2-LonSBm(P5A) was co-expressed.

### (2) Effect of LONRF2 on the Ubiquitination of TDP43

To examine whether LONRF2 ubiquitinates the abnormally structured TDP43 protein, an in vivo ubiquitination assay was performed in the same manner as in (1) above, except that FLAG-TDP43 was used instead of FLAG-hnRNP M1. The results are shown in FIG. 13. In A549 cells overexpressing TDP43, misfolded TDP43 proteins induced by sodium arsenite treatment were specifically ubiquitinated only when LONRF2-WT was co-expressed (FIG. 13). When LONRF2-RINGm (C4A) or LONRF2-LonSBm (P5A) was co-expressed, ubiquitination of misfolded proteins was not observed.

### (3) Binding of Misfolded Proteins to LONRF2

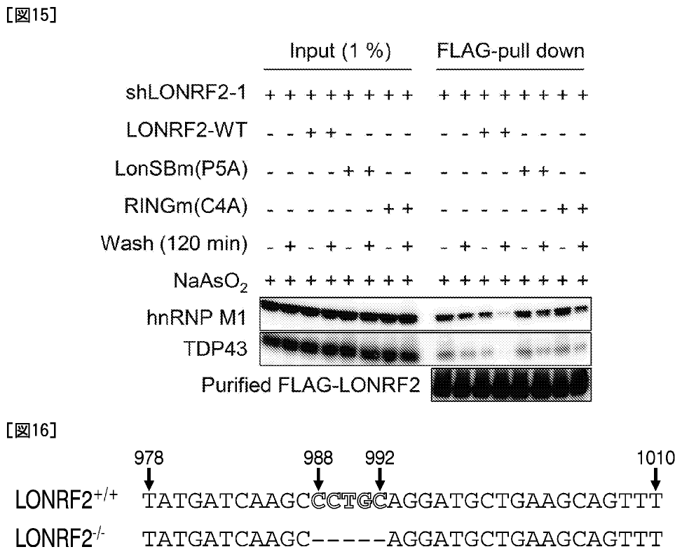

A549 cells expressing FLAG-LONRF2-WT, FLAG-LONRF2-RINGm (C4A), or FLAG-LONRF2-LonSBm (P5A) were incubated with or without 1 mM sodium arsenite for 30 minutes and then lysed in a lysis buffer containing a protease inhibitor and a deubiquitinase inhibitor. The resulting cell lysates were immunoprecipitated with anti-FLAG M2 affinity gel and then immunoblotted with an anti-FLAG antibodies. The results are shown in FIG. 14. As a result, both LONRF2-WT and LONRF2-RINGm(C4A), which contain the LonSB domain of wild-type LONRF2, bound to both the misfolded TDP43 protein and the misfolded hnRNP M1 protein generated by sodium arsenite treatment (FIG. 14).

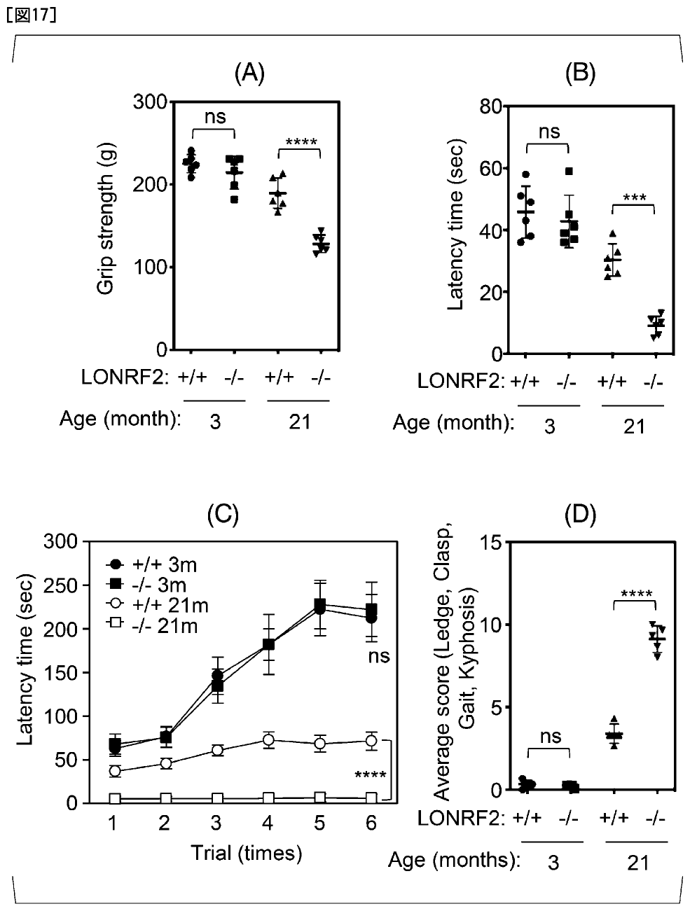

To determine whether LONRF2 reduces the abundance of misfolded TDP43 and hnRNP M1 in cells, a similar experiment was performed in A549 cells lacking endogenous LONRF2. Specifically, A549 cells expressing Dox-inducible shLONRF2-1 with LONRF2-WT, LONRF2-RINGm (C4A), or LONRF2-LonSBm (P5A) were cultured in the presence of Dox (1 mg/mL) for 48 hours and then incubated in the presence of 1 mM sodium arsenite for 30 minutes. FLAG-LONRF2 pull-down assays were performed on cell lysates of cells treated with sodium arsenite or cell lysates washed with sodium arsenite and incubated in PBS for 120 minutes. The results are shown in FIG. 15. In cells co-expressing LONRF2-WT, both hnRNP M1 and TDP43 were detected in cell lysates from cells that were not washed after sodium arsenite treatment, but almost no hnRNP M1 or TDP43 was detected in cell lysates from cells that were washed after sodium arsenite treatment. This was presumed to be because misfolded proteins generated by sodium arsenite were digested and reduced by LONRF2-WT. In cells co-expressing LONRF2-RINGm(C4A) or LONRF2-LonSBm(P5A), no reduction in hnRNP M1 or TDP43 was observed after washing. Similar results were observed in MCF7 cells and AsPC-1 cells.

### Example 3

To investigate the physiological role of LONRF2 in the PQC network and the development of neurodegenerative diseases, LONRF2 knockout mice (LONRF2^{-/-}mice) were generated that have an allele with a 5-bp deletion in exon 2. FIG. 16 shows the alignment of the nucleotide sequences around the 5-bp deletion site in exon 2 of the LONRF2 gene in LONRF2^{+/+} mice (LONRF2-WT mice) and LONRF2^{-/-}mice (LONRF2-KO mice).

### (1) Generation of LONRF2-KO Mice

The experiments described in the present work followed the Guidelines for Animal Experiments of the Institute of Medical Science, the National Center for Geriatrics and Gerontology Animal Ethics Committee, or the University of Tokyo and the Institutional Laboratory Animal Care

LONRF2 knockout mice were developed by CRISPR/Cas9-mediated zygote genome editing. Guide RNA (gRNA) which recognizes the coding region of the exon was used as “Mm.Cas9.LONRF2.1.AD” which was purchased from IDT. TracrRNA, gRNA, and Cas9 protein were purchased from IDT, and frozen pronuclear stage C57BL/6J zygotes were purchased from Clea-Japan.

TAKE methods(Non Patent Literature 23) with slight modifications were used to introduce gRNA, tracrRNA, and Cas9 protein into intact zygotes. Frozen zygotes were thawed by CARD methods (Non Patent Literature 24) and placed in a chamber (CUY505P, NEPA GENE) which was filled with Opti-MEM (ThermoFisher Scientific) containing 100 ng/mL gRNA, 200 ng/mL tracrRNA, and 200 ng/mL Cas9 protein. After electroporation, the zygotes were cultured in KSOM (Merck-Millipore) at 37° C. under 5% CO₂ in humid air conditions before embryo transfer. Embryos that developed to the two-cell stage on the next day of electroporation were transferred into the oviducts of pseudopregnant ICR female surrogates (supplied by Japan SLC) at 0.5-day postcoitum to obtain gene-manipulated offspring. Each surrogate and her transgenic descendants were bred onto a C57BL6/N background for at least four generations. To confirm genome editing, the lonrf2 genomic locus was amplified by PCR using KOD-FX neo (TOYOBO) employing a primer set flanking the gRNA-binding region, and sequences of PCR amplicons were analyzed. The gRNA sequence used for lonrf2 knockout and primer sequences used for lonrf2 genotyping were as follows. The gRNA sequence for PAM was aGG.

TABLE 5

| | Sequence | Seq. No. |
|---|---|---|
| gRNA | GAACTATGATCAAGCCCTGC | 23 |
| Forward primer for | GCTAACTTATGAATAAAATACCATGACC | 24 |
| lonrf2 genotyping | | |
| Reverse primer for | TGGAAACAATGTATTTTAAAATGCAGTC | 25 |
| lonrf2 genotyping | ATTTAT | |

LONRF2-KO mice were born without any apparent development abnormalities at normal Mendelian ratios, weighed the same as wild-type littermates, and appeared normal up to 18 months of age. LONRF2-KO mice then developed a gait abnormality.

### <Body Weight Measurement and Survival Rate Evaluation>

Each mouse was weighed early in the morning at the same time for consecutive 7 days. The mouse was gently picked up by its tail and placed inside a box for weighing at the age of 3-months and 21-months (n=6, males). The weighing room door was closed and the ambient noise was kept to a minimum to prevent undue excitement and unwanted escapees. For the survival rate evaluation, LONRF2^{+/+} mice (n=14, 8 males and 6 females) and LONRF2^{-/-}mice (n=11, 4 males and 7 females) were monitored weekly up to 136 weeks and the survival rate was recorded.

### (2) Analysis of Motor Function in LONRF2-KO Mice

The motor functions of young (3-month-old males, n=6) and old (21-month-old males, n=6) LONRF2-WT and LONRF2-KO mice were examined.

### <Grip Strength Test>

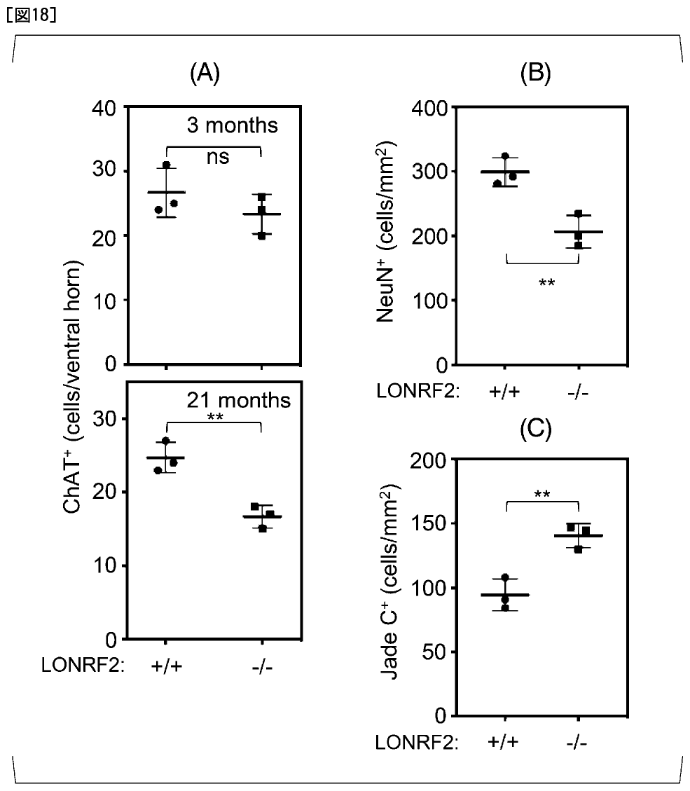

Grip strength of mouse was measured with a computerized instrument (BIOSEB), in which the mouse was gently pulled back until the grid was released and the maximal force (g) was recorded. The measurement results are shown in FIG. 17 (A). Data are shown as the mean±s.d. of three independent experiments. Statistical analysis was performed by one-way ANOVA with Dunnett's post-hoc test for multiple comparisons.

### <Wire Hang Test>

Each mouse was suspended on the wire by its front paws and the latency prior to falling was recorded. The measurement results are shown in FIG. 17 (B). Data are shown as the mean±s.d. of three independent experiments. Statistical analysis was performed by one-way ANOVA with Dunnett's post-hoc test for multiple comparisons.

### <Rota-Rod Test>

A Rota-Rod apparatus (Muromachi, “MK-630B Single LANE ROTAROD”) was used to measure motor coordination and balance. Each mouse was placed on a rotating rod with a constant rotation of 10-40 rpm and its latency in falling (measured in seconds) was recorded at a rotation of 40 rpm. Each animal was tested in the same manner for 6 consecutive days. The measurement results are shown in FIG. 17 (C). Data are shown as the mean±s.d. of three independent experiments. Statistical analysis was performed by two-way ANOVA with Dunnett's post-hoc test for multiple comparisons.

### <Composite Phenotype Scoring>

A composite phenotypic scoring system for mouse models of cerebellar ataxia was used to summarize the results from four individual assays (hind limb clasping, ledge walk, gait, and kyphosis) into one composite score. Each assay was scored on a scale of 0 to 3, with a total score ranging from 0 (unaffected) to 12 (severe). Data are presented as the mean±s.d. of three independent experiments. Statistical analysis was performed by one-way ANOVA with Dunnett's multiple comparison post-hoc test.

### <Ledge Test>

Each mouse was placed on the ledge of the cage and its ability to walk along the ledge was evaluated. The evaluation was done using a score on a scale from 0 to 3 as follows:
0: Walked normally along the ledge and then lowered itself into the cage using its paws.
1: Lost balance but appeared coordinated.
2: Lost balance and landed on its head on lowering itself into the cage.
3: Fell off the ledge.

### <Hind Limb Clasping>

Each mouse was lifted by the tail and hung upside-down for 10 seconds to observe the degree to which the hind limbs remained extended. The evaluation was done using a score ranged from 0 to 3 as follows.
0: Normal consistent extension.
1: Only one hindlimb remained extended for more than 5 seconds.
2: Both hindlimbs were retracted toward the abdomen for more than 5 seconds.
3: Both hindlimbs touched the abdomen for more than 5 seconds.

### <Gait>

Each mouse was placed on a flat surface with its head facing away from the investigator, and its gait was recorded. The evaluation was done using a score ranged from 0 to 3 as follows.
0: The body was supported on all limbs.
1: A tremor appeared.
2: Severe tremors and lowered pelvis.
3: Impaired physical mobility.

### <Kyphosis>

Each mouse was placed on a flat surface and its walking ability was evaluated. The evaluation was done using a score ranged from 0 to 3 as follows.
0: Straight spine.
1: Mild kyphosis.
2: Persistent but mild kyphosis.
3: Severe kyphosis.

As a result, the LONRF2-KO mice had scores comparable to those of the LONRF2-WT mice at 3 months of age, and had normal motor function. The composite scores of both mice at 3 months of age were also close to 0 (FIG. 17(D)). At 21 months of age, the LONRF2-KO mice did not lose weight, but developed significant age-dependent motor disorders, such as reduced grip strength, shortened time to fall, and impaired motor learning in the rotarod test (FIG. 17(A)-(C)). Furthermore, the LONRF2-KO mice at 21 months of age showed a significantly higher composite score than the LONRF2-WT mice (FIG. 17(D)). Accordingly, LONRF2-KO mice displayed a shorter life span than control LONRF2-WT mice.

### (3) Analysis of Motor Function in LONRF2-KO Mice

The age-dependent motor deficits and high composite scores suggest neurodegenerative changes in the spinal cord, cortex, and/or cerebellum. Therefore, the brain and spinal cord were subjected to immunohistochemical analysis. Single cell analysis of a dataset from mouse spinal cord revealed that LONRF2 predominantly expressed in cholinergic and excitatory neurons.

### <Immunohistochemical Staining>

Each mouse was anesthetized by placing in a closed container with 2 mL isoflurane and subsequently sacrificed to isolate the brain and the lumbar spinal cord tissue. Formalin-fixed paraffin-embedded sections were prepared from the brain and lumbar spinal cord tissues, respectively. Each tissue section was immunostained by incubation with the appropriate antibody or fluorescent dye. All tissue sections were co-stained with Hoechst or DAPI for nuclear staining. Immunofluorescently or immunohistochemically stained tissue sections were visualized and imaged using a confocal microscope (Zeiss, LSM710 NLO 2-photon) or a fluorescent microscope (Keyence, BZ-9000).

Immunostaining of each tissue section was performed using anti-calbindin antibody (Cell Signalling Technology, 13176), anti-NeuN antibody (Abcam, ab104224), anti-Ataxin2 antibody (Proteintech, 21776-1-AP), anti-phospho(409/410)-TDP43 antibody (Proteintech, 1078-2-AP), anti-G3BP1 antibody (Proteintech, 13057-2-AP), anti-ChAT antibody (Millipore, AB144P), Fluoro-Jade C Ready-to-Dilute Staining (FBS) antibody (FBS) . . . A kit (Biosensis, TR-100-FJ) was used.

### <Evaluation of the Neuronal Cell Number and the Stress Granule Level>

Neuronal degeneration in the brain (n=6, males) and lumbar spinal cord (n=3, males) was evaluated by counting ChAT-positive motor neuronal cells (spinal cord only), NeuN-positive neuronal cells, and Fluoro Jade C-positive degenerating neuronal cells per ventral horn or square millimeter. Two hundred cells were examined per section.

Quantitative analysis of stress granules of LONRF2^{-/-}mice was performed by evaluating positive Ataxin-2, G3BP1, and P-TDP43 inclusions in the brain (n=6, male) and lumbar spinal cord (n=3, male). Fluorescent dots represented stress granule inclusions. Two hundred cells were examined per section.

### <Quantification of Cerebellar Layer Thickness and the Purkinje Cell Number>

For molecular and granular layer measurements, layer thicknesses at 100 maicrometer intervals were measured in HE stained midsagittal sections using Image J software. The layer thickness for each section was obtained by averaging 20 measurements per section. Results were expressed as means±SD of 6 mice. To quantify Purkinje cells, brain tissue was stained with the antibody against the Purkinje cell specific protein, Calbindin. Midsagittal sections of comparable regions of 6 mice were used for cell counting. Using ImageJ to draw a segmented line along Purkinje cell soma (approximately a 30 mm length for each mouse), Purkinje cells were counted and the number of cells was divided by the length of the Purkinje cell layer.

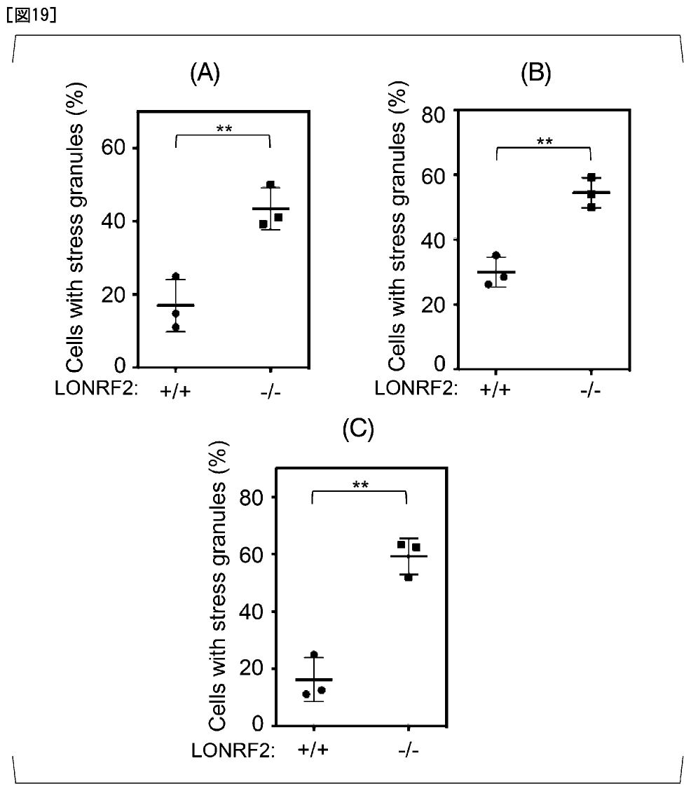

The results of measuring the number of ChAT-positive motor neurons per ventral horn in the lumbar spinal cord of 3-month-old and 21-month-old LONRF2-WT and LONRF2-KO mice are shown in FIG. 18(A). The results of measuring the number of NeuN-positive neurons per square millimeter in the lumbar spinal cord of 21-month-old LONRF2-WT and LONRF2-KO mice are shown in FIG. 18(B), and the results of measuring the number of Fluoro Jade C-positive degenerated neurons per square millimeter in the lumbar spinal cord of 21-month-old LONRF2-WT and LONRF2-KO mice are shown in FIG. 18(C). The percentages (%) of Ataxin 2-positive inclusion-containing cells, G3BP1-positive inclusion-containing cells, and phospho-TDP43-positive inclusion-containing cells per millimeter square in the lumbar spinal cord of 21-month-old LONRF2-WT mice and LONRF2-KO mice are shown in FIG. 19 (A) to (C), respectively.

Immunohistochemical analysis of the spinal cord revealed that the number of ChAT-positive neurons was similar in LONRF2-WT and LONRF2-KO mice at 3 months of age, but was significantly reduced in LONRF2-KO mice compared with LONRF2-WT mice at 21 months of age (FIG. 19(A)). Furthermore, ChAT-positive neurons in LONRF2-KO mice appeared abnormally shaped and shrunken. The number of NeuN-positive cells was reduced in LONRF2-KO mice compared with 21-month-old LONRF2-WT mice, and Fluoro Jade was significantly reduced in LONRF2-KO mice compared with 21-month-old LONRF2-WT mice. The number of C-positive cells increased (FIGS. 19(B) and (C)). The number of ataxin-2 and G3BP1 inclusion-positive cells, which are markers of components of stress granules, and the number of phospho-TDP43 inclusion-positive cells increased significantly (FIG. 19(A) to (C)). Ataxin-2 inclusions, G3BP1 inclusions, and phospho-TDP43 inclusions are all inclusions known to be involved in the onset of ALS.

Similar neurodegeneration was observed in the cerebral cortex and cerebellum of 21-month-old LONRF2-KO mice, with a decrease in NeuN-positive cells and an increase in Jade C-positive cells and inclusion-positive cells for Ataxin2, G3BP1, and phospho-TDP43. Furthermore, the number of Purkinje cells and the thickness of the granular and molecular layers were significantly decreased in 21-month-old LONRF2-KO mice compared with LONRF2-WT mice. No such neurodegeneration was observed in the striatum.

These results suggest that LONRF2 likely functions as a PQC ubiquitin ligase that degrades misfolded proteins such as TDP43 in vivo, and that loss of this function causes ALS-like neurodegenerative diseases and cerebellar ataxia.

While many model mice for ALS and SCD (spinocerebellar degeneration) develop neurodegeneration and ataxia relatively early, LONRF2-KO mice showed delayed symptoms at around 18 to 21 months of age. Because ALS and some types of SCD are delayed-onset diseases, LONRF2-KO mice are considered to be good models for these diseases and useful for drug screening.

(4) Effect of Ectopic Expression of LONRF2 on Motor Neurons Derived from iPS Cells in LONRF2-KO Mice

The neurological and cellular phenotypes of LONRF2-KO mice were consistent with the function of LONRF2 observed in cancer cells. Therefore, it was examined whether ectopic expression of LONRF2 could restore function to neurons lacking LONRF2.

First, primary fibroblasts from both LONRF2-WT and LONRF2-KO mice were infected with Sendai virus constructs encoding OCT4, SOX2, NANOG, and c-Myc to generate iPS cells. The generated iPS cells were evaluated by alkaline phosphatase staining. iPS cells generated from fibroblasts derived from LONRF2-KO mice (LONRF2^{-/-}iPS cells) proliferated at approximately the same rate as iPS cells generated from fibroblasts derived from LONRF2-WT mice (LONRF2^{+/+} iPS cells).

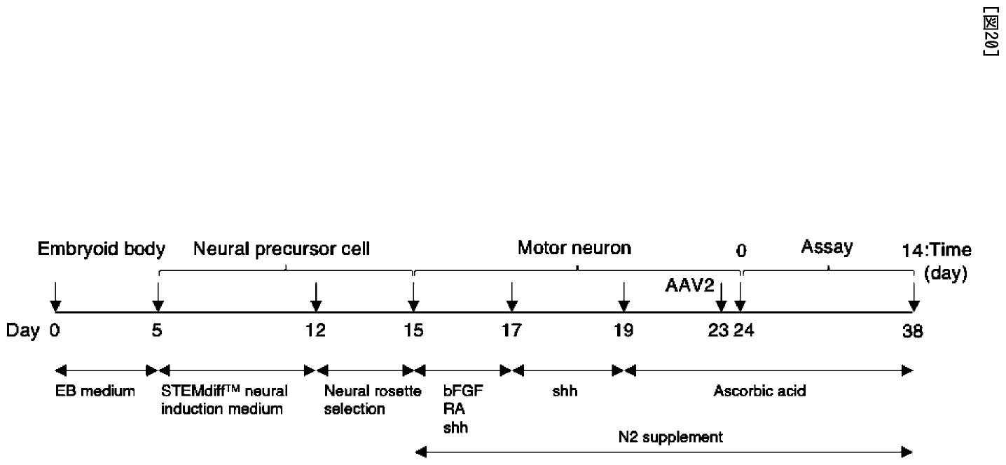

Next, each iPS cell was differentiated into a motor neuron by a 5-step method (Non Patent Literatures 25 and 26) in which the cells were cultured under the culture conditions shown in FIG. 20. First, the cells were cultured in EB medium for 5 days to differentiate into embryoid bodies, then cultured in neural induction medium “STEMDiff (registered trademark)” for 7 days, and then further cultured in differentiation induction medium “Neural rosette selection medium” for 3 days to differentiate into neural progenitor cells. Then, the neural progenitor cells were cultured for 2 days in a medium containing basic fibroblast growth factor (bFGF), retinoic acid (RA), and shh (Sonic Hedgehog) protein in N2 medium, cultured for 2 days in a medium containing only shh in N2 medium, and further cultured for 5 days in a medium containing ascorbic acid in N2 medium to differentiate into motor neurons. For the last 12 hours, the cells were cultured in a medium containing or not containing AAV-FLAG-LONRF2 (5×105 GC/mL), which is an adeno-associated virus vector (AAV) that contains a gene encoding FLAG-LONRF2. The cells were then cultured in N2 medium containing ascorbic acid for 14 days. When assessed by co-staining with Tuj 1 and ChAT, the efficiency of differentiation of LONRF2^{-/-}iPS cells into motor neurons was very high and was equivalent to that of LONRF2^{+/+} iPS cells.

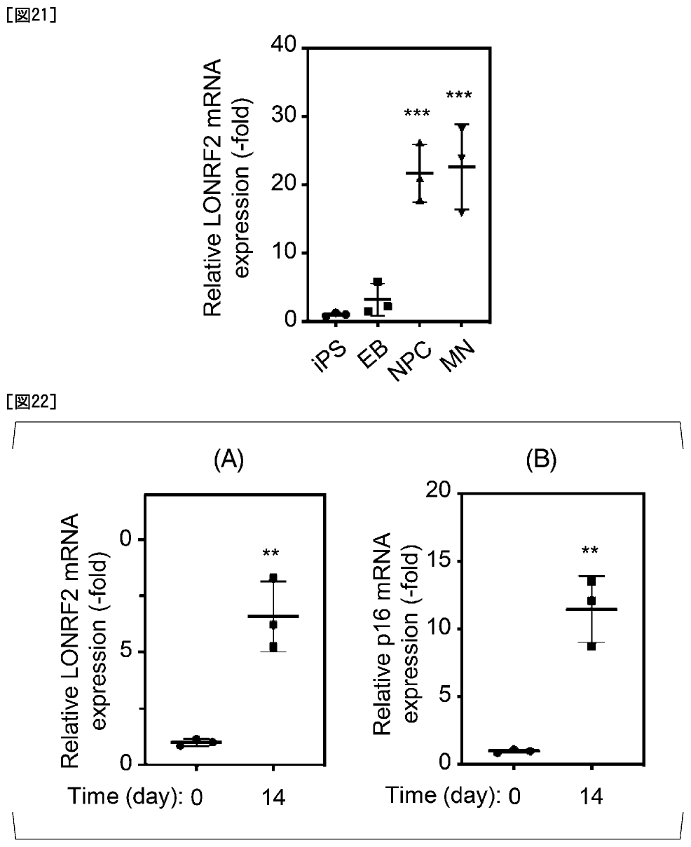

The expression level of LONRF2 at each differentiation stage from LONRF2^{+/+} iPS cells to motor neurons was examined by qPCR analysis. The relative amount of LONRF2 mRNA in cells at each differentiation stage is shown in FIG. 21. The results were consistent with the predominant expression of LONRF2 in mouse neurons. That is, the level of LONRF2 transcript was very low in iPS cells and embryoid bodies, but was dramatically induced in neuronal precursor cells and mature motor neurons. After differentiation into motor neurons, long-term culture for an additional 14 days increased the expression of both p16 and LONRF2 (FIGS. 22(A) and (B)). In FIGS. 21 and 22, data are expressed as the mean±s.d. of three independent trials. The data in FIG. 21 were analyzed by ANOVA and Dunnett's multiple comparison post-hoc test. Data in FIG. 22 were analyzed by ANOVA and unpaired two-tailed Student's t-test (*: p<0.05, **: p<0.01, ***: p<0.001, ****: p<0.0001).

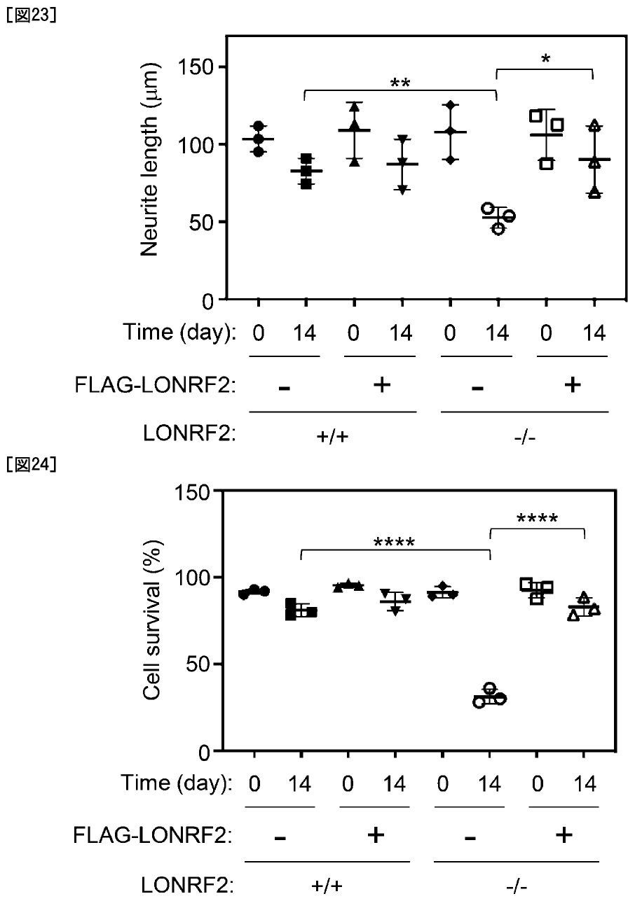

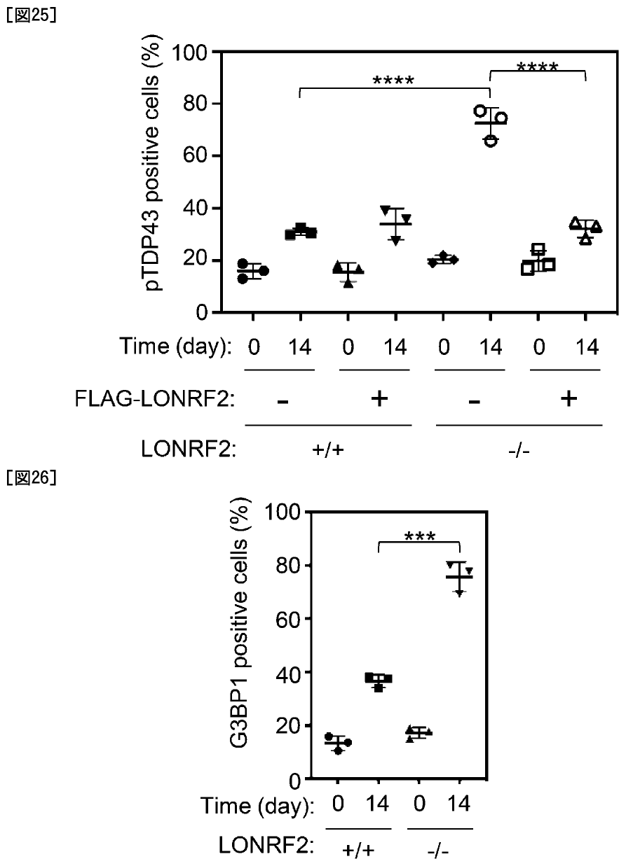

For motor neurons differentiated from LONRF2^{+/+} iPS cells and motor neurons differentiated from LONRF2^{-/-}iPS cells, the length of neurites (m) (FIG. 23), viability (ratio of TUNEL-negative cells:%) (FIG. 24), ratio of pTDP43-positive cells (%) (FIG. 25), and ratio of G3BP1-positive cells (%) (FIG. 26) were measured before culture (day 0) and after 14 days of culture. In FIG. 23 to 25, the column “−” in the “FLAG-LONRF2” column shows the results of cells not infected with AAV-FLAG-LONRF2, and the column “+” shows the results of cells infected with AAV-FLAG-LONRF2 to express FLAG-LONRF2 in the cells. FIG. 26 shows the results of cells not infected with AAV-FLAG-LONRF2. 23 to 26, data are expressed as the mean±s.d. of three independent trials. Data were analyzed by ANOVA and Dunnett's multiple comparison post-hoc test (*: p<0.05, **: p<0.01, ***: p<0.001, ****: p<0.000).

As shown in FIG. 23 to 26, the length of neurites was significantly shorter, the cell viability was significantly lower, and the ratio of pTDP43-positive cells and G3BP1-positive cells was significantly higher in LONRF2^{-/-}iPS cell-derived motor neurons. These were significantly restored to the same level as LONRF2^{+/+} iPS cell-derived motor neurons by expressing FLAG-LONRF2. Thus, LONRF2^{-/-}iPS cell-derived motor neurons showed shortened neurites, reduced viability after long-term culture, and accumulation of pTDP43 and G3BP1 after long-term culture, confirming that the neuronal abnormalities observed in LONRF2^{-/-}mice could be reproduced in LONRF2-deficient cultured motor neurons. In other words, it was suggested that the loss of LONRF2 in neurons directly caused cell death and the accumulation of misfolded proteins such as TDP43. Furthermore, these abnormalities observed in LONRF2-deficient cultured motor neurons were rescued by ectopic expression of LONRF2, indicating that loss of LONRF2 function is the cause of neurodegeneration and that it is treatable by restoring LONRF2 function (FIG. 23 to 26).

### Example 4

LONRF2-KO mice showed a neurodegenerative phenotype and cerebellar ataxia similar to ALS, suggesting that LONRF2 mutations may be involved in the development of neurodegenerative diseases such as ALS and SCD. Therefore, the relationship between LONRF2 and diseases was investigated using whole-sequence analysis data and population databases obtained from 41 familial ALS (FALS) patients, 446 sporadic ALS (SALS) patients, 1,163 healthy controls, and 158 SCD patients.

### <Reanalyzing Public Single-Cell RNA-Seq Dataset>s

For single-cell RNA-seq data, the data set was downloaded from the NCBI Gene Expression Omnibus (GEG) under the accession number GSE129788 for aging mouse brain and GSE161621 for adult mouse spinal cord. The reanalysis was performed using R (version 4.0) or Python (version 3.7) of supercomputer system SHIROKANE, Institute of Medical Science, University of Tokyo. t-SNE or UMAP clustering separated the different cell types, which was in accord with the original findings. Reanalysis of LONRF2 and ChAT expression levels was performed using the library of Seurat and Scanpy.

### <Genetic Analyses>

A total of 41 pedigrees with FALS and 446 patients with SALS in the Japanese series were recruited for clinical and molecular genetic studies. All the patients were diagnosed as having clinically definite, probable, laboratory-supported probable, or possible ALS on the basis of the El Escorial revised criteria. In addition, 1,163 unrelated healthy Japanese subjects with no reported history of ALS, frontotemporal dementia (FTD), or other neurodegenerative diseases were included as a source of control DNAs. Genomic DNA samples were obtained from all the participants with their written informed consent. Whole-exome sequence analysis was performed for a Japanese series of 41 FALS probands and 446 SALS patients, in whom causative variants have not been identified. To focus on rare variants, variants with a minor allele frequency (MAF)≥0.1% in any of the following population databases were excluded from the analysis, which included gnomAD East Asians (The Genome Aggregation Database; https://gnomad.broadinstitute.org/) (v. 2.1.1) and jMorp (Japanese Multi Omics Reference Panel; https://jmorp.megabank.tohoku.ac.jp; 8.3KJPN).

TABLE 6

| Genotypes | | FALS | SALS | Controls | ToMMo 8.3 KJPN | gnomAD-EAS |
|---|---|---|---|---|---|---|
| Exon | Variant | (n = 41) | (n = 446) | (n = 1163) | (MAF [%]) | (MAF [%]) |
| 1 | p.Ala20Gly | 0 | 0 | 2 | ND | 0 |
| 1 | p.Ala131Thr | 1 | 0 | 2 | 0.08 | 0.06 |
| 1 | p.Val167Met | 0 | 0 | 1 | ND | 0 |
| 9 | p.Val538Ile | 0 | 1 | 0 | ND | 0 |
| 9 | p.Ala585Val | 0 | 0 | 1 | ND | 0 |
| 10 | p.Val599Met | 0 | 2 | 0 | 0.07 | 0.03 |
| 11 | p.Ala665Val | 0 | 1 | 2 | 0.08 | 0.005 |
| 12 | p.Val705Met | 0 | 0 | 1 | ND | 0.006 |
| 12 | p.Ser721Leu | 0 | 1 | 0 | ND | 0.006 |

As a result of the analysis, several rare mutations were found in LONRF2. The mutations are shown in Table 4. No significant mutations in LONRF2 were found in SCD patients. Although not statistically significant, the frequency of patients with any LONRF2 variant was higher in FALS patients (2.44%) and SALS patients (1.12%) than in the control group collected by the inventors (0.77%) (Table 4). Many of these mutations were located in the essential domain of LonSB. In addition, some mutations were detected only in SALS patients and not in the control group.

We investigated whether these variants have the ability to bind and ubiquitinate misfolded TDP43. Specifically, we first prepared constructs of six single amino acid mutants in the LonSB domain (LONRF2-V538I, LONRF2-A585V, LONRF2-V599M, LONRF2-A655V, LONRF2-V705M, and LONRF2-S721L) from among those listed in Table 4 in the same manner as LONRF2-LonSBm(P5A).

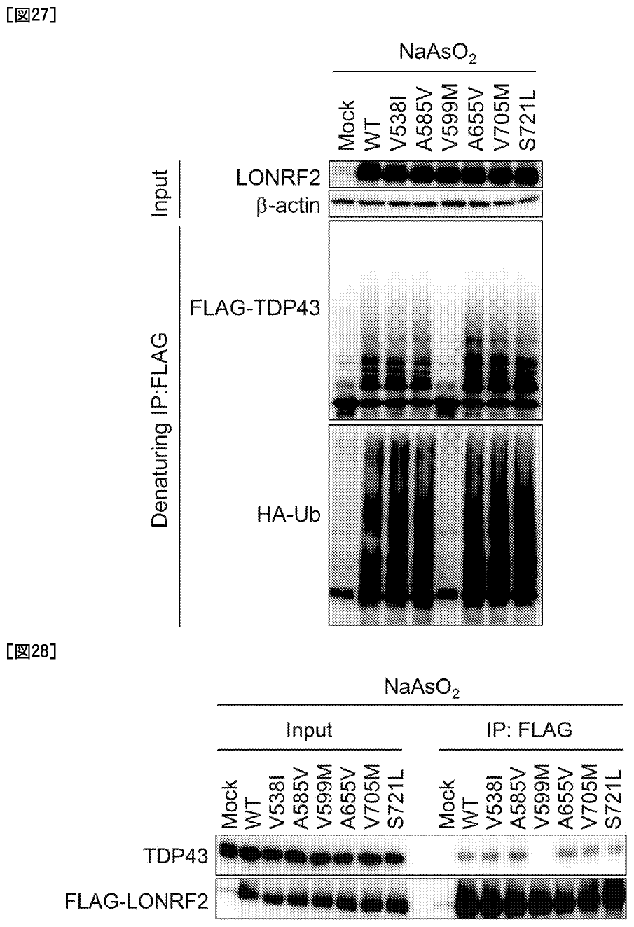

Next, to examine whether these mutants ubiquitinate the abnormally structured protein of TDP43, an in vivo ubiquitination assay was performed in the same manner as in Example 2. That is, HeLa cells co-expressing LONRF2-WT, LONRF2-V538I, LONRF2-A585V, LONRF2-V599M, LONRF2-A655V, LONRF2-V705M, LONRF2-S721L, or mock, FLAG-TDP43, and HA-Ub were treated with sodium arsenite (1 mM, 30 minutes) and then lysed under denaturing conditions. The obtained cell lysate was added with an equal amount of 2× denaturing IP buffer, incubated, and then immunoprecipitated with anti-FLAG M2 affinity gel and immunoblotted with anti-FLAG antibody. The results are shown in FIG. 27.

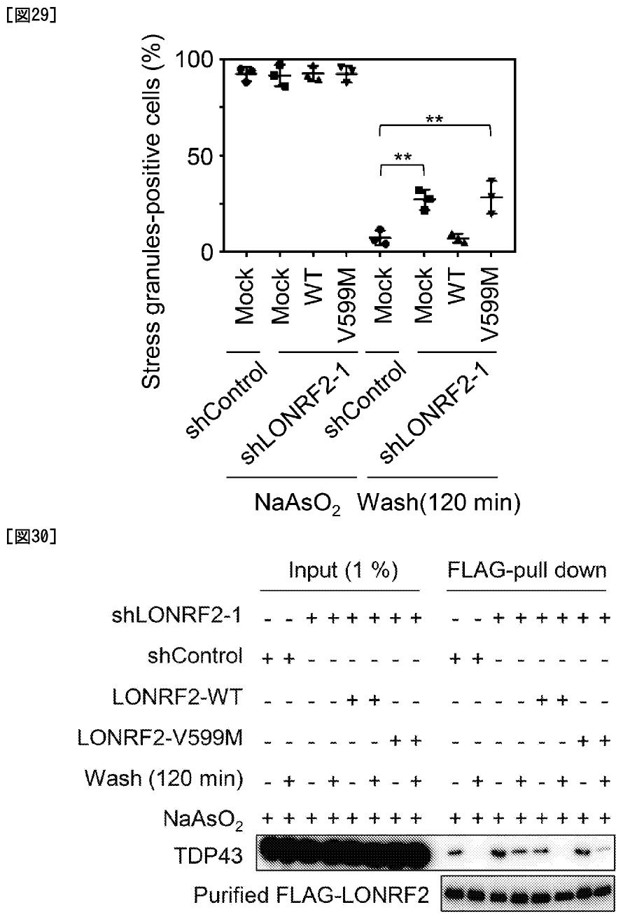

To examine whether the LONRF2 mutant binds to the abnormally structured protein of TDP43, immunoprecipitation and immunoblotting were performed in the same manner as in Example 2. Specifically, A549 cells expressing FLAG-LONRF2-WT, FLAG-LONRF2-V538I, FLAG-LONRF2-A585V, FLAG-LONRF2-V599M, FLAG-LONRF2-A655V, FLAG-LONRF2-V705M, FLAG-LONRF2-S721L, or mock were incubated in the presence or absence of 1 mM sodium arsenite for 30 minutes, and then lysed in a lysis buffer containing a protease inhibitor and a deubiquitinase inhibitor. The obtained cell lysate was subjected to immunoprecipitation with anti-FLAG M2 affinity gel, and then immunoblotting was performed using anti-FLAG antibody. The results are shown in FIG. 28.

As shown in FIGS. 27 and 28, only LONRF2-V599M (rs143848902) detected in two ALS patients was unable to bind to or ubiquitinate misfolded TDP43 in the presence of sodium arsenite, whereas the other mutants bound to and ubiquitinated misfolded TDP43 similarly to WT.

To examine the effect of LONRF2-V599M on the dynamics of stress granules, the proportion (%) of stress granule-positive cells among all cells (n=200) was determined in the same manner as in Example 1.

Specifically, d-Sen cells were prepared by introducing Dox-inducible shRNA (shLONRF2-1 or shControl). These d-Sen cells were cultured in the presence of Dox (1 mg/mL), and the cell lysates were subjected to Western blotting using an anti-LONRF2 antibody. It was confirmed that the expression level of LONRF2 was significantly reduced in the cells introduced with Dox-inducible shLONRF2-1 compared to the cells introduced with Dox-inducible shControl.

These d-Sen cells were then cultured in the presence of Dox (1 mg/mL) for 48 hours, incubated in the presence of 1 mM sodium arsenite for 30 minutes, and then washed in PBS for 120 minutes. After washing, the cells were subjected to fluorescent immunocytochemical staining using anti-G3BP1 antibody in the same manner as in Example 1 to examine the percentage (%) of stress granule-positive cells containing 5 or more G3BP1-positive foci in the total cells (n=200). The results are shown in FIG. 29. Data are shown as the mean±s.d. of three independent experiments. Statistical analysis was performed by one-way ANOVA with Dunnett's post hoc test for multiple comparisons. As shown in FIG. 29, expression of LONRF2-V599M in cells depleted of endogenous LONRF2 did not restore the impaired decomposition process of stress granules. Other single amino acid mutants that were able to bind to the misfolded protein of TDP43 were able to rescue the impaired stress granule disassembly process, similar to LONRF2-WT.

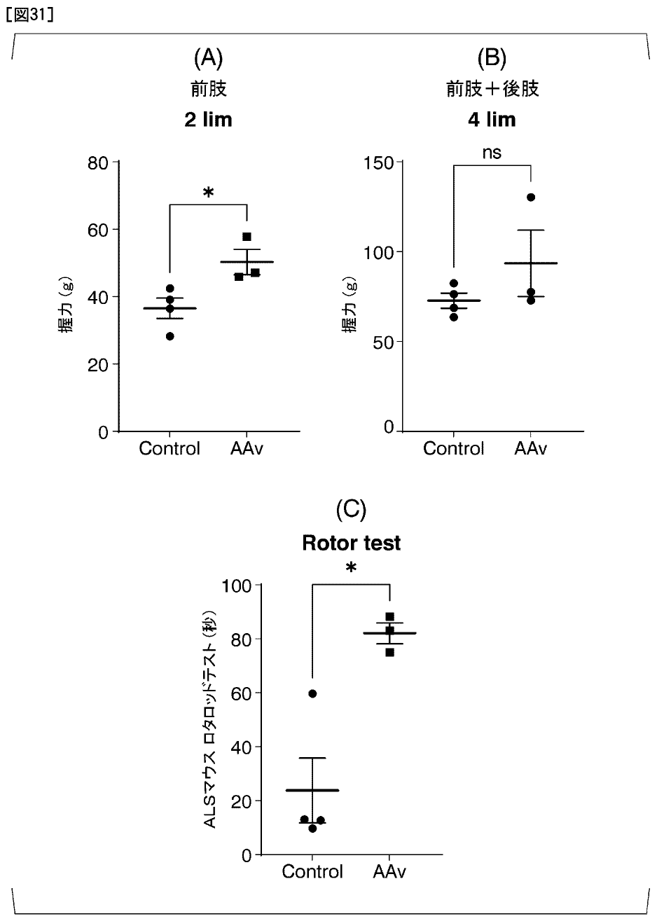

To examine whether LONRF2-V599M reduces the amount of misfolded TDP43 present in cells, an experiment similar to that in Example 2 was performed with A549 cells lacking endogenous LONRF2. Specifically, A549 cells expressing Dox-inducible shLONRF2-1 or Dox-inducible shControl and LONRF2-WT or LONRF2-V599M were cultured in the presence of Dox (1 mg/mL) for 48 hours, and then incubated in the presence of 1 mM sodium arsenite for 30 minutes. FLAG-LONRF2 pull-down assay was performed on cell lysates of cells treated with sodium arsenite or cell lysates treated with sodium arsenite and washed with PBS for 120 minutes. The results are shown in FIG. 30. As shown in the Figure, LONRF2-WT reduced the abundance of misfolded TDP43 in A549 cells after washing with sodium arsenite, whereas LONRF2-V599M failed to reduce the amount of misfolded protein.

These results indicate that LONRF2-V599M is a mutant that has lost at least the functions of binding to and ubiquitination of misfolded proteins in vitro, degradation of stress granules, and reduction of misfolded proteins in cells.

### Example 5

It was examined whether the pathology would improve by expressing LONRF2 in an ALS model. As the ALS model, an SOD1-G93AALS mouse model (purchased from Jackson Laboratory) was used as in the method of Miyoshi et al. (Non Patent Literature 27). Expression of LONRF2 was performed by infecting the mice with AAV-FLAG-LONRF2, which was prepared by incorporating a gene encoding FLAG-LONRF2 used in Example 3 into AAV. As a comparative control, AAV-EGFP, which was prepared by incorporating an EGFP gene into AAV, was used.

Of seven 5-week-old SOD1-G93AALS mouse models, three were administered 1.2×10¹² vg of AAV-FLAG-LONRF2, and the remaining four were administered an equal amount of AAV-EGFP via tail vein injection. After about 15 weeks from administration, the grip strength of the front and back limbs of each mouse was measured, and a rotor rod test was also performed.

The grip strength of the front and back limbs was measured using an electronic pull strain gauge (1027DSM, manufactured by Columbus Instruments). Three measurements were taken per mouse, and the average value was used for statistical analysis. These experiments were performed in a blinded manner. The rotor rod test was performed in the same manner as in Example 3.

Table 7 shows information such as the sex of each mouse. The results of the measurement of the front limb grip strength of each administration group are shown in FIG. 31 (A), the results of the measurement of the front and rear limb grip strength are shown in FIG. 31 (B), and the results of the rotarod test are shown in FIG. 31 (C). In the Figures and tables, “Control” represents the group administered with AAV-EGFP, and “AAv” represents the group administered with AAV-FLAG-LONRF2. As shown in FIG. 31 (A) to (C), the ALS mice expressing LONRF2 had improved grip strength and the results of the rotarod test were also improved. These results suggest that the pathology of ALS can be improved by increasing the expression level of LONRF2, and that gene therapy to express LONRF2 is effective in treating ALS.

TABLE 7

| | Ex. | Birth | Sex | BW (g) | Sac Day | Age (w) |
|---|---|---|---|---|---|---|
| 1 | Cont | Jul. 30, 2022 | f | 20.3 | Dec. 17, 2022 | 20 |
| 2 | | Jul. 30, 2022 | f | 19.9 | Dec. 17, 2022 | 20 |
| 3 | | Aug. 8, 2022 | m | 24.0 | Dec. 20, 2022 | 19 |
| 4 | | Aug. 8, 2022 | f | 21.9 | Dec. 20, 2022 | 19 |
| 5 | AAV | Aug. 8, 2022 | m | 27.9 | Dec. 20, 2022 | 19 |
| 6 | | Aug. 8, 2022 | f | 22.1 | Dec. 20, 2022 | 19 |
| 7 | | Aug. 8, 2022 | f | 22.4 | Dec. 20, 2022 | 19 |

### SEQUENCE LISTING

[symbol]

1. A method for detecting structurally abnormal proteins, the method comprising:
detecting a structurally abnormal protein in distinction from a normal protein by binding the structurally abnormal protein to an agent for detecting structurally abnormal proteins,
wherein an active ingredient of the agent for detecting structurally abnormal proteins is a polypeptide having structurally abnormal proteins-binding site or a functional nucleic acid for expressing the polypeptide in host cells, and
wherein the structurally abnormal proteins-binding site is:
(A) a polypeptide consisting of the amino acid sequence represented by SEQ ID NO:2, or
(B) a polypeptide consisting of an amino acid sequence having 90% or more sequence identity with the amino acid sequence represented by SEQ ID NO:2 and having binding activity to structurally abnormal proteins.

2. (canceled)

3. The method for detecting structurally abnormal proteins of claim 1, wherein:
the polypeptide having binding activity to structurally abnormal proteins does not bind to a wild-type protein of firefly luciferase,
the polypeptide having binding activity to structurally abnormal proteins has binding activity to an R188Q/R261Q double mutant protein of firefly luciferase,
the wild-type protein of firefly luciferase has the amino acid sequence represented by SEQ ID NO:3, and
the mutant protein of firefly luciferase has the amino acid sequence represented by SEQ ID NO:4.

4. (canceled)

5. The method for detecting structurally abnormal proteins of claim 1, wherein the polypeptide having the structurally abnormal protein-binding site is:
(A1) a polypeptide having the amino acid sequence represented by SEQ ID NO:1, or
(B1) a polypeptide having an amino acid sequence having 90% or more sequence identity with the amino acid sequence represented by SEQ ID NO:1 and having binding activity to structurally abnormal proteins and ubiquitin ligase activity.

6-7. (canceled)

8. The method for detecting structurally abnormal proteins of claim 1, wherein the agent for detecting structurally abnormal proteins is administered to a patient with a disease in which the structurally abnormal protein accumulates in the body, or to an individual at high risk of developing the disease, for the treatment or prevention of the disease.

9. The method for detecting structurally abnormal proteins of claim 8, wherein the disease is a neurodegenerative disease.

10. The method for detecting structurally abnormal proteins of claim 9, wherein the neurodegenerative disease is amyotrophic lateral sclerosis.

11. The method for detecting structurally abnormal proteins of claim 1, wherein the structurally abnormal protein is a misfolded protein.

12. A transgenic animal that has a deletion of the LONRF2 gene or has a mutation that reduces the function of the LONRF2 gene, and is used as a model for an amyotrophic lateral sclerosis.

13. The transgenic animal of claim 12, wherein the mutation is a V599M mutation.

14. A cell obtained from the transgenic animal of claim 12.

15. A method for evaluating the risk of developing a disease, the method comprising:
typing the genotype of rs143848902 of a human subject; and
evaluating the human subject's risk of developing a disease in which abnormal proteins accumulate in the body based on the typing of the genotype of rs143848902,
wherein the human subject is evaluated as having a high risk of developing the disease when the genotype of rs143848902 is the ATG type.

16. The method for evaluating the risk of developing the disease of claim 15, wherein the disease is amyotrophic lateral sclerosis.

17. (canceled)

18. The method for detecting structurally abnormal proteins of claim 1, wherein:
the polypeptide having structurally abnormal proteins-binding site has a ubiquitin ligase active site, and
structurally abnormal proteins are reduced in the cell into which the abnormal protein detection agent has been introduced.
